(19) United States
(12) Patent Application Publication
(10) Pub. No.: US 2021/0187114 A1
(43) Pub. Date: Jun. 24, 2021

(54) NOVEL LINKER, PREPARATION METHOD, AND APPLICATION THEREOF

(71) Applicant: Genequantum Healthcare (Suzhou) Co., LTD., Suzhou (CN)

(72) Inventors: Gang QIN, Suzhou (CN); Tan CHUBING, Suzhou (CN); Yuan JINDUO, Suzhou (CN); Lu JIANG, Suzhou (CN)

(73) Assignee: Genequantum Healtcare (Suzhou) Co., LTD., Suzhou (CN)

(21) Appl. No.: 17/135,877

(22) Filed: Dec. 28, 2020

Related U.S. Application Data
(62) Division of application No. 14/787,700, filed on Dec. 28, 2015, now abandoned.

(30) Foreign Application Priority Data
Apr. 28, 2013 (CN) 201310171802.4
Mar. 17, 2014 (CN) 201410111810.4

(51) Int. Cl. A61K 47/68; A61K 47/65; A61K 47/64; A61K 31/5365; A61K 39/395; C07K 7/06; C07K 16/30; C12N 15/113

(52) U.S. Cl. CPC A61K 47/6803; A61K 47/6889; A61K 47/65; A61K 47/64; A61K 47/6885; C07K 1/04; A61K 39/39558; C07K 7/06; C07K 16/30; C12N 15/1137; A61K 31/5365

(57) ABSTRACT

Provided in the present invention is a linker and a preparation method thereof, wherein one end of the linker may covalently link a small molecule compound and the like and the other end may specifically and covalently link a targeting substance site under the action of Sortase enzyme. The linker of the present invention can be used to prepare a targeting drug conjugate.

### TECHNICAL FIELD

The present invention belongs to the biopharmaceutical and biotechnology fields, particularly a new kind of coupling linkers, their preparation methods, and their applications in the coupling of small molecules, nucleic acids and analogs and imaging agents to either the N or C terminal of proteins or polypeptides. The linkers and the corresponding conjugation methods disclosed herein are used in the preparation of targeting tumor drugs (i.e. ADCs), targeting imaging diagnosis agents and highly efficient cell specific delivery agents.

### BACKGROUND

Targeted delivery of small molecules, proteins, peptides, nucleic acids, nucleic acid analogs and imaging agents into specific cell type or tissue is critical and challenging in biomedical research as well as in clinical diagnosis and treatment. One of the most important applications is the development of highly specific antibody-drug conjugates (ADCs) for targeted cancer therapy. So far FDA has approved two ADCs: Adcetris in 2011 for treatment of Hodgkin Lymphoma (Seattle Genetics) and Kadcyla in 2013 for treatment of invasive breast cancer (Roche).

Antibody-drug conjugates (ADCs) are the next generation of monoclonal antibody therapy which combined the targeting function of antibodies with the high efficiency of the traditional cytotoxins. ADCs are composed of three components: a cell specific antibody, a linker and a cytotoxin. The antibody determines the target cell type; Linker is the most important technology in the design of ADC drug which controls the targeting release of the drug; the cytotoxins are compounds which cause cell death, induce apoptosis or inhibit cell viability. The key technology of ADC drug is the design of the coupling strategy, it is critical for the drug targeting. Many technologies are currently available, including chemical ligation, non-natural amino acid modification of the andibody, and enzyme catalyzation etc.(please refer to the technologies developed by Seattle Genetics, Immunogene, Mersana, Ambrx, Pfizer, etc). However, all these technologies face similar problems such as heterogeneous coupling sites and number, complicate processing protocols and such. The heterogeneity of ADCs will seriously affect the pharmaceutical kinetics, drug stability and reproducibility. Site-specific, highly homogenous coupling is the future direction of the ADC drugs.

Nucleic acids and nucleic acid analogs such as antisense, siRNA, displayed some special advantages in cancer therapy, which might play a key role in the next generation of bio-therapeutics. However, many nucleic acid/nucleic acid analog drugs under phase

II/III clinical trials are coated by lipid and other nano materials which lack target specificity. It is reported that antibody can be used as siRNA targeting agent, but the siRNA and antibody are not covalently jointed, which made the reported siRNA-antibody complex highly heterogeneous, resulting in unpredictable and suboptimal pharmacokinetic, poor stability and pharmaceutical efficacy (Yao Y-D et al., Sci Transl Med.2012, 4(130):130ra48), thus prevented its application in clinical. Clearly, covalent conjugation of siRNA and other therapeutic molecules with antibody in a site specific manner would be ideal.

RNA interfering experiment in cell culture has become an essential technique in biomedical research. Conventional delivery strategy is transfection reagents based (commercialized by Invitrogen, Roche). These reagents are sometimes toxic to cells and the effectiveness of these reagents varies greatly with cell types. Therefore, the development of highly efficient, feasible delivery methods is of high demanding.

Sortases are a group of transpeptidases generated by Gram-positive bacteria. Their high specificity and efficiency in protein ligation make sortases a very good tool for site-specific ligations of protein-peptide, protein-nucleic acid analog, protein-glyco and the labeling of living cells. The application of sortase in site specific labeling of proteins have been reported (Mohlmann et al, Chembiochem. 2011,12(11):1774-80; Madej MP et al, Biotechnol Bioeng. 2012 , 109(6):1461-70; Swee LK et al, Proc Natl Acad Sci U S A. 2013, 110(4):1428-33;). Genetically engineered sortases were also reported, provided even more variety of catalytic properties. However, the application of sortases to antibody-drug, antibody-cytotoxin, antibody-siRNA and antibody-oligonucleotide conjugation has not been realized technically, mainly due to technical challenges in linker design and conjugation procedure.

### SUMMARY

The purpose of the present invention is to provide a novel coupling system to solve some of the problems encountered in the preparation of ADCs, targeting nucleic acid drugs, targeting tracer diagnostic agents and efficient cell delivery agents.

1. The linkers

The present invention provides a series of linkers with bifunctional groups. In particular, it consists of three areas: a Protein Conjugation Area (PCA), a Linker Area (LA) and a Chemical Conjugation Area (CCA) as shown in the following structures:

PCA1−(LA) a−CCA1 (I)

or

CCA2−(LA) a-PCA2 (II).

When the targeting moiety is a protein or antibody, PCA is a short peptide sequence, representing the sequence of a substrate of a natural Sortase (including Sortases A, B, C, D, L. and Plantarum etc., see patent US20110321183A1) or a gene engineered Sortase (e.g., Chen I et al, Proc Natl Acad Sci USA. 2011, 108 (28): 11399-404). In particular:

Formula (I) presents the first category of linker, wherein PCA1 is a suitable receptor substrate sequence of Sortase, which composed of polyglycine (Gly)n (n is typically 1-100), the C-terminal α-carboxylic group of which is used to couple with the LA or directly to CCA1; the PCA1 in formula (I) may also be other suitable receptor substrate for Sortase A, such as polyalanine (Ala)n or a copolymer of Glycine and Alanine.

Formula (II) presents the second category of linker, wherein PCA2 is a suitable donor substrate sequence of Sortase. In particular, the substrate sequence for Staphylococcus aureus Sortase A is LPXTG; for Staphylococcus aureus Sortase B it is NPQTN; for Bacillus anthracis Sortase B it is NPKTG, and for Streptococcus pyogenes Sortase A it is LPXTG; for Streptomyces coelicolor Sortase subfamily5 it is LAXTG, while for Lactobacillus plantarum Sortase it is LPQTSEQ.

The general formula of PCA2 is: X1X2X3TX4X5X6, where X1 represents leucine (Leu) or asparagine (Asn), X2 represents proline (Pro) or alanine (Ala), X3 represents any amino acid, X4 represents threonine (Thr), X5 represents glycine (Gly), serine (Ser) or asparagine (Asn), X6 represents any amino acid or absent. PCA2 is connected to LA (or directly to CCA2) through its N-terminal α-amino group.

It must be pointed out that when the targeting moieties are peptides, the structure of PCA in both formula (I) and formula (II) may either be designed as described above or the sequence of the peptide itself.

The amino acids in PCA1 and PCA2 as shown in formula (I) and formula (II) are all in the L-type except glycine.

LA is a linkage between PCA and CCA, a is 0 or 1, meaning LA may or may not exist. The structure of LA is shown in the following formula:

NH2-R1-P-R2-(C═O)—OH.

On one hand, P represents polyethylene glycol unit with the formula of (OCH2CH2) m, where m is 0 or an integer of 1-1000; R1, R2 represents H, a linear alkyl group having 1 to 6 carbon atoms; a branched or cyclic alkyl group with 3 to 6 carbon atoms; a linear, branched or cyclic alkenyl or alkynyl group having 2-6 carbon atoms; LA in the above formula can be covalently linked to PCA and CCA via its amino and carboxyl groups at either ends.

On the other hand, P represents a peptide with 1-100 residues; R1, R2 may represent H, a linear alkyl group having 1 to 6 carbon atoms; a branched or cyclic alkyl group with 3 to 6 carbon atoms; a linear, branched or cyclic alkenyl or alkynyl group having 2-6 carbon atoms; LA in the above formula can be covalently linked to PCA and CCA via its amino and carboxyl groups at either ends.

Examples of linear alkyl include methyl, ethyl, propyl, butyl, pentyl and hexyl group. Examples of branched or cyclic alkyl having 3-6 carbon atoms include isopropyl, isobutyl, tertiary butyl, pentyl, hexyl, cyclopropyl, cyclobutyl, cyclopentyl and cyclohexyl group.

Examples of linear alkenyl having 2 to 6 carbon atoms include ethenyl, propenyl, butenyl, pentenyl, hexenyl. Examples of a branched or cyclic alkenyl having 2 to 6 carbon atoms include isobutenyl, isopentenyl, 2-methyl-1-pentenyl, 2-methyl-2-pentenyl.

Examples of the linear alkynyl having 2 to 6 carbon atoms include ethynyl, propynyl, butynyl, pentynyl, hexynyl. Examples of branched or cyclic alkynyl having 2 to 6 carbon atoms include 3-methyl-1-butyne, 3-methyl-1-pentynyl, 4-methyl-2-hexynyl.

A CCA must have appropriate functional groups to form amide, disulfide, thioether, thioester, hydrazone, ester, ether or urethane bond with small molecules, a nucleic acids, or tracer molecules. Preferred functional groups include, but not limited to: N-succinimidyl esters and N-sulfosuccinimidyl ester, p-nitrophenyl ester, di-nitrophenyl and pentafluorophenyl ester, etc. suitable for reaction with an amino group to form an amide bond; maleimide group(suitable to react with a thiol group), carboxylic acid chloride (to react with a thiol group); pyridyldithio and nitropyridyl dithio, to form a disulfide bond with another thiol group; and haloalkyl or haloacetyl to react with a thiol group to form thiol ether; isocyanate group to react with a hydroxyl group to form isothiocyanates; carboxyl group to form an ester bond with a hydroxyl, or an amino group to form an amide, etc. A CCA also contains a functional group such as: a carbonyl group to form an oxime bond with alkoxyamine; an azide or alkynyl group to perform Cu (I) catalyzed and promoted strain Huisgen 1,3-dipolar cycloaddition (the ‘Click’ reaction); an electron-deficient tetrazine or a strained alkene to perform an inverse electron demand hetero Diels-Alder (HDA) reaction), and other functional groups to perform Michael reaction, metathesis reaction, transition metal elements catalyzed cross-coupling reactions, free radical oxidative couplings, oxidative coupling, acyl-transfer reactions and photo click reactions (Kim CH et al, Curr Opin Chem Biol 2013 Jun; 17 (3.): 412-9).

Type I of the preferred CCA1 contains a peptide sequence with 1-200 residues, wherein at least one residue is lysine. The N-terminal a-amino group of this peptide is connected to LA or directly to PCA1 via an amide bond, the C-terminal of this peptide is either an acid or an amide. Based on the said number of drug loaded, the c-amino group of lysine is either directly coupled to a suitable bifunctional molecule to introduce coupling groups as maleimide, pyridyl dithio, haloalkyl\haloacetyl or isocyanate. Preferably, the α-, or/and ε-amino group of lysine is further reacted with more lysines, the α-, or/and ε-amino group of the said “more lysines” may also be used to introduce more coupling groups. By repeating this step, many lysines may be incorporated and a branched structure of lysine is obtained which allows the introduction of functional groups of 1-1000. Alternatively, other amino acid(s) may also be incorporated into the branched structure of Lysine. For example, a glycine may be coupled to the α- or ε-amino of lysine, the α-amino of this glycine is coupled to another lysine. As required, the number and type of amino acids incorporated between the lysines may be one or more. The said other amino acid is further coupled with other functional linkers to increase the number and type of functional groups. These other amino acids may be any amino acids. For example, the said other amino acids incorporated may be a cysteine, the said cysteine is connected to an appropriate coupling agents through its side chain thiol groups. Alternatively, any unnatural amino acid(s) may be incorporated between any two of the branched lysines, for example, a hydrocarbon group or a cylic hydrocarbon group containing reactive groups capable of covalently connected with a carboxyl or an amino group of an amino acid on its both ends. Preferably, a bifunctional crosslinking agent which incorporated a maleimide, a pyridyl dithio, a haloalkyl, a haloacetyl, or an isocyanate functional group into the CCA1 includes but not limited to: N-Succinimidyl 4-(N-maleimidomethyl) cyclohexane-1-carboxylate(SMCC), the “long chain” analog of SMCC N-[alpha-maleimidoacetoxy] Succinimide ester(AMAS), N-gamma-Maleimidobutyryl-oxysuccinimide ester(GMBS), 3-MaleiMidobenzoic acid N-hydroxysucciniMide ester(MBS), 6-maleimidohexanoic acid N-hydroxysuccinimide ester(EMCS), N-Succinimidyl 4-(4-Maleimidophenyl) butyrate(SMPB), Succinimidyl 6-[(beta-maleimido-propionamido) hexanoate (SMPH), N-Succinimidyl 11-(maleimido) undecanoate(KMUS); Those coupling reagents comprising N-hydroxysuccinimide-(polyethylene glycol)n-maleimide bifunctional groups (SM (PEG) n), where n represents 2,4,6,8,12 or 24; those haloacetyl-based crosslinking reagents include Succinimidyl (4-iodoacetyl) aminobenzoate(SIAB), Succinimidyl iodoacetate(SIA), N-Succinimidyl bromoacetate(SBA) and N-Succinimidyl 3-(Bromoacetamido) propionate(SBAP); Cross-linking agents comprises dithiopyridyl groups are N-Succinimidyl 3-(2-Pyridyldithio) propionate(SPDP), Sulfosuccinimidyl-6-[(a-methyl-(a-(2-pyridyldithio) toluamido] hexanoate(S-LC-SMPT), sulfosuccinimidyl-6-[3-(2-pyridyl-dithio)-propionamido] hexanoate(S-LC-SPDP). The preferred linkers meeting the above requirements are shown in FIGS. 1-12, but not limited thereto.

Another type of the preferred CCA1 structures containing peptides with 1-200 residues having amides formed by condensation reaction between a-amino groups and carboxyl groups, contains at least one cysteine. The N-terminal a-amino group of this CCA1 may form an amide bond with LA (or directly with PCA1), the carboxyl terminus of the peptide is —COOH or —CONH2. The side chain thiol group of cysteine is connected to the bi-functional crosslinking agent which has maleimide, dithiopyridyl, haloacetyl or haloalkyl group. Such preferred crosslinking agents are divided into 2 groups. Group 1 is applied to react with nucleic acids, tracer molecules, and other small molecules which contain primary amino groups. Those preferred bifunctional crosslinking agents which connected to the cysteine side chain thiol group include but not limited to: N-Succinimidyl 4-(N-maleimidomethyl) cyclo hexane-1-carboxylate(SMCC), SMCC “long chain” analog N-[alpha-maleimidoacetoxy] Succinimide ester(AMAS), N-gamma-Maleimidobutyryl-oxysuccinimide ester(GMBS), 3-Maleimidobenzoic acid N-hydroxysuccinimide ester(MBS), 6-maleimidohexanoic acid N-hydroxysuccinimide ester(EMCS), N-SucciniMidyl 4-(4-MaleiMidophenyl) butyrate(SMPB), Succinimidyl 6-[(beta-maleimidopropionamido) hexanoate (SMPH), Succinimidyl 4-(N-maleimidomethyl) cyclohexane-1-carboxy-(6-amidocaproate)(LC-SMCC), N-Succinimidyl 11-(maleimido) undecanoate(KMUS), those comprising N-hydroxysuccinimide-(polyethyleneglycol) n-maleimide bifunctional crosslinking agents (SM (PEG) n), where n presents 2,4,6,8,12 or 24; and those containing dithiopyridyl groups including but not limited to: N-Succinimidyl 3-(2-Pyridyldithio) propionate(SPDP), sulfosuccinimidyl-6-[(a-methyl-a-(2-pyridyldithio)toluamido]hexanoate(S-LC-SMPT), Sulfosuccinimidyl-6-[3-(2-pyridyldithio)-propionamido]hexanoate(S-LC-SPDP), Succinimidyl (4-iodoacetyl) aminobenzoate(SIAB), Succinimidyliodoacetate(SIA), N-Succinimidylbromoacetate(SBA) and N-Succinimidyl 3-(Bromoacetamido) propionate(SBAP). Group 2 may react with the hydroxyl group of small molecules, nucleic acids, tracer molecules. The bifunctional cross-linking agent connected to the cysteine side chain thiol group includes but not limited to: N-(p-Maleimidophenyl isocyanate)(PMPI). The examples of the preferred linkers that meet the above requirements are shown in FIGS. 13-18, but not limited thereto.

A third preferred type of CCA1 contains a peptide with 1-200 residues, wherein at least one chemically reactive residue is of non-natural amino acid. The chemically reactive residues of non-natural amino acid may be directly incorporated or on to the side chain of an amino acid (via amine, carboxyl, thiol, hydroxyl etc). Those chemically reactive groups may covalently couple with a suitable small molecule, a nuclei acid or a tracer molecule through the formation of oxime, Cu(I)-catalyzed and strain-promoted Huisgen 1,3-dipolar cycloadditions (‘Click’ reaction), inverse electron demand hetero Diels-Alder (HDA) reactions, Michael reactions, metathesis reactions, transition metal catalyzed cross-couplings, radical polymerizations, oxidative couplings, acyl-transfer reactions, and photo click reactions

The N-terminal α-amino of this peptide form an amide bond with LA (or directly with PCA1), the C-terminus of the peptide is —COOH or —CONH2. Based on the expected number of coupling, corresponding number of non-natural amino acids are incorporated. Preferred examples of the linkers meeting the above requirements are shown in FIG. 19-25, but not limited thereto.

The above designed features of CCA1 may be used individually or in combination, which means different functional groups may be included in one CCA1 which may allow the covalently coupling of different small molecules, nucleic acid(s), and/or tracer molecules.

One preferred type of CCA2 of linker II contains a peptide sequence with 1-200 residues, forming amide bonds through the condensation of α-amino and carboxyl groups, wherein at least one residue is lysine. The C-terminal α-carboxyl group of this peptide is connected to LA or directly to PCA2 via an amide bond. Based on the number of drug loaded, the ε-amino group of lysine is either directly coupled to a suitable bifunctional molecule to introduce coupling groups as maleimide, pyridyl dithio, haloalkyl\haloacetyl or isocyanate, or formed an amide through its ε-amino group and the a-carboxyl group of another lysine so as to from a branched structure, and further the α- and ε-amino groups of the branched lysine may incorporate maleimide, pyridyl dithio, haloalkyl\haloacetyl or isocyanate with a suitable bi-functional crosslinking agent. The functional groups introduced by the later method are doubled. Optionally, the α-, or/and ε-amino group of lysine is further reacted with more lysines, the α-, or/and ε-amino group of the said “more lysines” may further be used to introduce more coupling groups. By repeating this step, many lysines may be incorporated and a branched structure of lysines is obtained which allow the introduction of functional groups of 1-1000. Alternatively, one or more other amino acids or one or more non-amino acid may also be incorporated into the branched structure of Lysine as mentioned above. Preferably, the said bifunctional crosslinking agents which may incorporate a maleimide, a pyridyl dithio, a haloalkyl, a haloacetyl, or an isocyanate functional group into the CCA2 include but not limited to: N-Succinimidyl 4-(N-maleimidomethyl) cyclohexane-1-carboxylate(SMCC), the “long chain” analog of SMCC N-[alpha-maleimidoacetoxy] Succinimide ester(AMAS), N-gamma-Maleimidobutyryl-oxysuccinimide ester(GMBS), 3-MaleiMidobenzoic acid N-hydroxysucciniMide ester(MBS), 6-maleimidohexanoic acid N-hydroxysuccinimide ester(EMCS), N-Succinimidyl 4-(4-Maleimidophenyl) butyrate(SMPB), Succinimidyl 6-[(beta-maleimido-propionamido) hexanoate (SMPH), N-Succinimidyl 11-(maleimido) undecanoate(KMUS); Those coupling reagents comprising N-hydroxysuccinimide-(polyethyleneglycol)n-maleimide bifunctional groups (SM (PEG) n), where n represents 2,4,6,8,12 or 24; those haloacetyl-based crosslinking reagents include Succinimidyl (4-iodoacetyl) am inobenzoate(SIAB), Succinimidyl iodoacetate(SIA), N-Succinimidyl bromoacetate(SBA) and N-Succinimidyl 3-(Bromoacetamido) propionate(SBAP); Cross-linking agents comprises dithiopyridyl groups are N-Succinimidyl 3-(2-Pyridyldithio) propionate(SPDP), Sulfosuccinimidyl-6-[(a-methyl-(a-(2-pyridyldithio) toluamido] hexanoate(S-LC-SMPT), sulfosuccinimidyl-6-[3-(2-pyridyl-dithio)-propionamido] hexanoate(S-LC-SPDP). The preferred linkers meeting the above requirements are shown in FIGS. 26-31, but not limited thereto.

Another type of the preferred CCA2 structure of linker II contains peptides with 1-200 residues, forming amide bonds through the condensation of α-amino and carboxyl groups, wherein at least one residue is cysteine. The C-terminal α-carboxyl group of this CCA2 may form an amide bond with LA (or directly with PCA2). The side chain thiol group of cysteine is connected to a bi-functional crosslinking agent containing a maleimide, dithiopyridyl, haloacetyl or haloalkyl group. Such preferred crosslinking agents are divided into 2 groups. Group 1 is applied to react with nucleic acids, tracer molecules, and other small molecules which contain a primary amino group. Those preferred bifunctional crosslinking agents which connected to the cysteine side chain thiol group include but not limited to: N-Succinimidyl 4-(N-maleimidomethyl) cyclo hexane-1-carboxylate(SMCC), SMCC “long chain” analog N-[alpha-maleimidoacetoxy] Succinimide ester(AMAS), N-gamma-Maleimidobutyryl-oxysuccinimide ester(GMBS), 3-Maleimidobenzoic acid N-hydroxysuccinimide ester(MBS), 6-maleimidohexanoic acid N-hydroxysuccinimide ester(EMCS), N-SucciniMidyl 4-(4-MaleiMidophenyl) butyrate(SMPB), Succinim idyl 6-[(beta-maleimidopropionamido) hexanoate (SMPH), Succinimidyl 4-(N-maleimidomethyl) cyclohexane-1-carboxy-(6-amidocaproate)(LC-SMCC), N-Succinimidyl 11-(maleimido) undecanoate(KMUS), those comprising N-hydroxysuccinimide-(polyethylene glycol) n-maleimide bifunctional crosslinking agents (SM (PEG) n), where n presents 2, 4, 6, 8,12 or 24; and those containing dithiopyridyl groups including but not limited to: N-Succinim idyl 3-(2-Pyridyldithio) propionate(SPDP), sulfosuccinimidyl-6-[(a-methyl-a-(2-pyridyldithio)toluamido]hexanoate(S-LC-SMPT), Sulfosuccinim idyl-6-[3-(2-pyridyldithio)-propionamido] hexanoate(S-LC-SPDP), Succinimidyl (4-iodoacetyl) aminobenzoate(SIAB), Succinimidyl iodoacetate(SIA), N-Succinimidyl bromoacetate(SBA) and N-Succinimidyl 3-(Bromoacetamido) propionate(SBAP). Group 2 may react with the hydroxyl group of small molecules, nucleic acids, tracer molecules. The bifunctional cross-linking agents connected to the cysteine side chain thiol group include but not limited to: N-(p-Maleimidophenyl isocyanate) (PMPI).

A third preferred type of CCA2 of linker II contains a peptide with 1-200 residues, forming amide bonds through the condensation of α-amino and carboxyl groups, wherein at least one residue is of non-natural amino acid.

The chemically reactive residues of non-natural amino acid may be directly incorporated or on to the side chain of an amino acid (via amine, carboxyl, thiol, hydroxyl etc). Those chemically reactive groups may covalently couple with a suitable small molecule, a nuclei acid or a tracer molecule through the formation of oxime, Cu(I)-catalyzed and strain-promoted Huisgen 1,3-dipolar cycloadditions (‘Click’ reaction), inverse electron demand hetero Diels-Alder (HDA) reactions, Michael reactions, metathesisreactions, transition metal catalyzed cross-couplings, radical polymerizations, oxidative couplings, acyl-transfer reactions, and photo click reactions. The α-carboxyl group of this peptide forms an amide bond with LA (or directly with PCA2). Based on the desired number of coupling, corresponding number of non-natural amino acids are incorporated. Preferred examples of the linkers meeting the above requirements are shown in FIGS. 32-35, but not limited thereto.

The above features of CCA2 may be used individually or in combination, which means different functional groups may be included in one CCA2 which may allow the covalently coupling of different small molecules, nucleic acid(s), and/or tracer molecules.

In particular, the structures of PCA1 and PCA2 shown in FIG. 1-35 are designed based on the optimized substrate sequence of Staphylococcus aureus Sortase A. The PCA1 and PCA2 of this invention covers all the substrates of any Sortase A, no matter it is a native enzyme, an optimally screened enzyme, or gene-engineered enzyme. The structures of PCA1 and PCA2 can also be native or modified peptides which have targeting feature(s).

The linker in the present invention may be synthesized using standard solid-phase peptide synthesis protocols, based on Fmoc chemistry (which is well known to those skilled in the art).

A general protocol is as follows:

(1) Choice of resin: Solid phase synthesis is carried out using Wang or Rink amide resin which is pre-loaded with the C-terminal amino acid of a linker. Based on the choice of resin, the C-terminus of the peptide is either carboxylic acid or an amide.

(2) Swelling resin: the amount of resin used is calculated based on the final product required, the difficulty of the synthesis and a purification loss. The resin is added DCM or DMF (N, N, -Dimethylformamide), soaking for 30 min.

(3) Fmoc removal: the DMF used for soaking the resin was drained. 20% piperidine in DMF is added, and the reaction is bubbled for 10min, drained, and repeated the solution again for 15 min, to totally remove the Fmoc from the a-amino, revealing the reactive amino group in order to connect to the carboxyl group of the next amino acid. Filtration, DCM wash twice, DMF three times, followed by ninhydrin assay, resin should be in dark blue.

(4) Coupling of the amino acid: 2-5 equivalent of the next amino acid is dissolved in DMF, to the solution was added an appropriate amount of coupling reagent DIC (Diisopropylcarbodiimide)/HBTU (2-(1H-Benzotriazole-1-yl)-1,1,3,3-tetramethyl-aminium hexafluorophosphate) These were all added into a reaction column and react under nitrogen stirring for 2 h. The resin should be nearly colorless with ninhydrin test when the reaction is completed. Afterwards, the resin is washed twice with DCM, and then three times with DMF.

(5) Blocking the reactive sites on the resin: In order to ensure the purity of the final product, a small amount of un-reacted amino group must be capped. 20% of acetic anhydride is added to the resin for 10-30min under nitrogen stirring. After completion of the reaction the resin is washed twice with DCM, and then three times with DMF.

(6) Monitoring the coupling progress: a small amount of sample is taken after each coupling step to check the free amino group by ninhydrin test. If the resin is colorless, the reaction has been completed. If the resin is purple or black (positive reaction), indicating there is still unreacted amino group, the coupling reaction should be repeated.

(7) Coupling the rest of the amino acids: Repeat steps 3-6 until the sequence is completed coupled. Synthesis process may also be used to incorporate other suitable intermediate (e.g., polyethylene glycol analogue).

(8) Solid phase coupling of functional groups: a particular side-chain protecting group (eg, the ε-amino of lysine with Dde) is orthogonal deprotected and then coupled with a suitable bifunctional crossing reagent (This step is optional, which can also be carried out after step 9 “cleavage” under certain circumstances)

(9) Cleavage: When the last amino acid is coupled and the Fmoc group removed, the resin is dried, added to a 50 ml flask. A cleaving mixture made of TFA/phenol/H2O ratio/EDT/TIS (85/5/5/3/2) is added and stirred at 0-5° C. for 2 h. The resin is filtered, and cold ether of 30× volume is added to the TFA solution, precipitate is collected and freeze-dried to give crude peptide or analogue.

(10) Purification and mass spectrometry characterization: The crude peptide is dissolved in acetonitrile/water solution, analyzed by reverse phase HPLC, and a preparative gradient determined. The purified peptide was analyzed again by HPLC, and more than 95% purity components collected. The molecular weight is confirmed by ES-MS. If necessary, NMR.

2. The small molecules, nucleic acids or tracer molecules

The small molecules of the present invention mainly refer to cytotoxic drugs, including any compound that can cause cell death, inducing apoptosis or inhibit cell viability. The cytotoxic drugs include, but not limited to: microtubule inhibitors such as paclitaxel and its derivatives, auristatins derivatives such as MMAE, MMAF, etc., maytansine and derivatives, epothilones analogues, vinca alkaloids such as vinblastine, vincristine, vindesine, vinorelbine, vinflunine, vinglycinate, anhydrovinblastine, dolastatin and analogues, halichondrin B, meturedopa, uredopa, camptothecine and its derivatives, bryostatin, Callystatin, Melphalan, nitrosoureas such as carmustine, fotemustine, Lomustine, Nimustine, uramustine, ranimustine, neocarzinostatin, dactinomycin, porfiromycin, anthramycin, azaserine, esorubicin, bleomycin, carabicin, idarubicin, nogalamycin, carzinophilin, carminomycin, dynemicin, esperamicin, epirubicin, mitomycin, olivomycin, peplomycin, puromycin, marcellomycin, rodorubicin, streptonigrin, ubenimex, zorubicin, methotrexate, denopterin, pteropterin, trimetrexate, thiamiprine, fludarabine, thioguanine and other purine analogs; pyrimidine analogs such as ancitabine, azacitidine, cytarabine, dideoxyuridine, 5′-deoxy-5-fluorouridine, enocitabine, floxuridin, calusterone, drostanolone, epitiostanol, mepitiostane, testolactone, aceglatone, aldophosphamide glycoside, aminolevulinic acid, bisantrene, edatrexate, colchicinamide, diaziquone, eflornithine, elliptinium acetate, lonidamine, mitoguazone, mitoxantrone, pentostatin, betasizofiran, spirogermanium, tenuazonic acid, triaziquone, verracurin A, roridin A, anguidine, dacarbazine, mannomustine, mitolactol, pipobroman, DNA topoisomerase inhibitors, flutamide, nilutamide, bicalutamide, leuprorelin acetate and Goserelin, protein kinases and proteasome inhibitors.

Tracer molecules of the present invention include but not limited to, fluorescent molecules (such as TMR, Cy3, FITC, Fluorescein, etc.) or radioactive nuclides.

The nucleic acids of the present invention include, but not limited to single-stranded and/or double-stranded DNAs, RNAs, nucleic acid analogues. Preferred nucleic acid molecule is siRNA.

3. The coupling intermediates

The small molecules, nucleic acids and tracer molecules disclosed in this invention all have a mercapto, hydroxy, carboxy, amino, alkoxyl-amino, alkynyl, azide, tetrazine or other functional groups in a preferred position. The small molecules, nucleic acids or tracer compounds are covalently attached to the claimed linker I or II, resulted in coupling intermediates of the following formula:

PCA1−(LA)ₐ−CCA1−Payloadₕ (III)

or

Payloadₕ−CCA2−(LA)ₐ−PCA2 (IV),

in which payload refers specifically to a small molecule, a nucleic acid or a tracer molecule,

a is 0 or 1,

h is the number of small molecules, nucleic acids or tracer molecules that linked to each linker, is from 1 to 1000. When h>1, the payloads are same or different.

In order to obtain the coupling intermediates, the linkers are synthesized first on a solid phase, purified and characterized and then coupled with small molecules, nucleic acids or tracer molecules under appropriate conditions. The coupling is carried out in an organic or aqueous solution at appropriate pH, according to the features of the functional groups to be linked. The resulted coupling intermediate is analyzed by reverse phase HPLC, based on the retention time and purity to determine the gradient for preparative HPLC. The purified coupling intermediate is characterized via UPLC-MS. Melting point, and NMR are determined if necessary.

Under certain circumstances, the coupling intermediates are made in an one-step protocol, that means the small molecules, nucleic acids or tracer molecules may be coupled to the linker on a solid phase, before the cleavage. The intermediate is then cleaved from the resins, totally deprotected. The resulted coupling intermediate is analyzed by reverse phase HPLC, based on the retention time and purity to determine the gradient for preparative HPLC. The purified coupling intermediate is characterized via UPLC-MS. Melting point, and NMR are determined if necessary.

4.The targeting moieties

The targeting moieties included in the present invention are preferably recombinant antibodies and antibody analogs (e.g., Fab, ScFv, minibody, diabody, nanobody, etc.). It may also be non-antibody proteins, including but not limited to, interferons, lymphokines (e.g., Interleukins), hormones (e.g., insulin), growth factors (e.g., EGF, TGF-α, FGF, and VEGF), and may also be targeting peptides (native peptides, such as peptide GPCR ligands, or unnatural amino acid modified peptides).

Based on the structural information of the targeting proteins, the N or C-terminus of the sequence is chosen as coupling site to ensure that the protein function is not influenced.

When a payload is coupled to the N-terminus of a targeting protein, an intermediate with a structure of formula(III) is used. In order to ensure sortase catalyzed site-specific coupling, a sortase substrate sequence, i.e. polyglycine, is engineered into the N-terminus of the targeting protein. In order to obtain such an N-terminal modified protein, a suitable substrate sequence of another particular protease (e.g., TEV enzyme, thrombin, etc.) is incorporated after the N-terminal Methionine of the protein, followed by the Sortase substrate sequence, which is released after treatment of this said protease. Or alternatively, a suitable Sortase substrate sequence such as polyglycine is incorporated right after the N-terminal methionine, and the sortase substrate sequence is released by a host cell endogenous or engineered methionyl aminopeptidase to take off the N-terminus methione.

When the payload is coupled to the N-terminus of a peptide, polyglycine is directly incorporated into the N-terminal of the peptide during the synthesis.

When a payload is coupled to the C-terminus of a targeting protein, an intermediate with a structure of formula(IV) is used. In order to achieve highly specific coupling, a suitable substrate sequence of sortase or other more preferred enzymes must be incorporate into the C-terminus of the protein. For Sortase A, this C-terminal sequence is LPXTGG, X may be any natural amino acid.

When the payload is couple to the C-terminus of a peptide, the sortase substrate sequence is incorporated into the C-terminal of the peptide during the synthesis.

5. The targeting mioeties and coupling intermediates are linked together in a site-specific manner to form the final conjugate

The targeting moieties (such as antibodies, proteins or peptide) as described in section 4 and a particular coupling intermediate as described in section 3 are mixed, a natural sortase or more preferably a selected sortase is added to linked the two sections together in a site specific way. The preferred buffer contains NaCI at a concentration of 1-1000 nM, Ca²⁺ at a concentration of 0-50 mM, and at pH 5-10. The preferred reaction temperature is 4-45° C. and reaction time is 10 min-20 h. SDS-PAGE, HPLC and/or ESI-MS are used to analyze the coupling efficiency, and the crude conjugate product is purified by gel shift FPLC, or preparative HPLC.

The ligation reaction is illustrated in FIG. 36, the resulted targeting drug conjugates are shown in formula (V) or (VI):

T−PCA1−(LA)ₐ−CCA1−payloadₕ (V)

Payloadₕ−CCA2−(LA)ₐ−PCA2-T (VI),

wherein:

T refers to a targeting moiety,

Payload refers to a small molecule, a nucleic acid or a tracer molecule,

a is 0 or 1,

h is the number of the small molecule, nucleic acid or tracer molecule coupled to each linker, h is an integer of 1-1000. When h>1, the payloads may be same or different.

### DESCRIPTION OF THE DRAWINGS

FIG. 1 A general structure of linker 1 (n=1-100, X is OH or NH2)

FIG. 2 A general structure of linker 2 (n=1-100, X is OH or NH2)

FIG. 3 A general structure of linker 3 (n=1-100, m=0, 1-1000, X is OH or NH2)

FIG. 4 A general structure of linker 4 (n=1-100, m=0, 1-1000, X is OH or NH2)

FIG. 5 A general structure of linker 5 (n=1-100, m=0, 1-1000, X is OH or NH2)

FIG. 6 A general structure of linker 6 (n=1-100, m=0, 1-1000, X is OH or NH2)

FIG. 7 A general structure of linker 7 (n=1-100, m=0, 1-1000, X is OH or NH2)

FIG. 8 A general structure of linker 8 (n=1-100, m=0, 1-1000, X is OH or NH2)

FIG. 9 A general structure of linker 9 (n=1-100, m=0, 1-1000, X is OH or NH2)

FIG. 10 A general structure of linker 10 (n=1-100, m=0, 1-1000, X is OH or NH2)

FIG. 11 A general structure of linker 11 (n=1-100, m=0, 1-1000, X is OH or NH2)

FIG. 12 A general structure of linker 12 (n=1-100, m=0, 1-1000, X is OH or NH2)

FIG. 13 A general structure of linker 13 (n=1-100, X is OH or NH2)

FIG. 14 A general structure of linker 14 (n=1-100, X is OH or NH2)

FIG. 15 A general structure of linker 15 (n=1-100, m=0, 1-1000, X is OH or NH2)

FIG. 16 A general structure of linker 16 (n=1-100, m=0, 1-1000, X is OH or NH2)

FIG. 17 A general structure of linker 17 (n=1-100, X is OH or NH2)

FIG. 18 A general structure of linker 18 (n=1-100, m=0, 1-1000, X is OH or NH2)

FIG. 19 A general structure of linker 19 (n=1-100, X is OH or NH2)

FIG. 20 A general structure of linker 20 (n=1-100, X is OH or NH2)

FIG. 21 A general structure of linker 21 (n=1-100, X is OH or NH2)

FIG. 22 A general structure of linker 22 (n=1-100, X is OH or NH2)

FIG. 23 A general structure of linker 23 (n=1-100, m=0, 1-1000, X is OH or NH2)

FIG. 24 A general structure of linker 24 (n=1-100, m=0, 1-1000, X is OH or NH2)

FIG. 25 A general structure of linker 25 (n=1-100, m=0, 1-1000, X is OH or NH2)

FIG. 26 A general structure of linker 26 (X is OH or NH2)

FIG. 27 A general structure of linker 27 (m=0, 1-1000, X is OH or NH2)

FIG. 28 A general structure of linker 28 (X is OH or NH2)

FIG. 29 A general structure of linker 29 (X is OH or NH2)

FIG. 30 A general structure of linker 30 (X is OH or NH2)

FIG. 31 A general structure of linker 31 (X is OH or NH2)

FIG. 32 A general structure of linker 32 (X is OH or NH2)

FIG. 33 A general structure of linker 33 (n=1-100, m=0, 1-1000, X is OH or NH2)

FIG. 34 A general structure of linker 34 (X is OH or NH2)

FIG. 35 A general structure of linker 35 (m=0, 1-1000, X is OH or NH2)

FIG. 36 The preparation process of antibody-dugs and antibody-siRNA conjugates

FIG. 37 The chemical structure of linker 1

FIG. 38 The UPLC profile of linker 1

FIG. 39 The ES I-MS profile of linker 1

FIG. 40 The UPLC profile of maysteine derivative DM1

FIG. 41 The ES I-MS profile of maysteine derivative DM1

FIG. 42 The structure of a coupling intermediate made of maysteine derivative DM1

FIG. 43 The UPLC-MS profile of an coupling intermediate made of maysteine derivative DM1

FIG. 44 The chemical structure of linker 26

FIG. 45 The HPLC profile of linker 26

FIG. 46 The ES I-MS of linker 26

FIG. 47 The structure of a coupling intermediate of GAPDH siRNA-linker 26

FIG. 48 The coupling efficiency of GAPDH siRNA with linker 26, checked by SDS PAGE 1: GAPDH siRNA; 2: the coupling intermediate GAPDH siRNA-linker 26

FIG. 49 The structure of coupling product: GAPDH siRNA-linker 26-GFP

FIG. 50 The coupling efficiency of GAPDH siRNA-linker 26 with GFP checked by native PAGE 1: GAPDH siRNA-linker 26; 2: 0 min, 3: 60 min, 4 120 min, *: final product siRNA-GFP; **: the coupling intermediate GAPDH siRNA-linker 26

FIG. 51 The structure of linker 2

FIG. 52 The HPLC profile of linker 2

FIG. 53 The ESI-MS of linker 2

FIG. 54 The structure of linker 3

FIG. 55 The HPLC profile of linker 3

FIG. 56 The ES I-MS of linker 3

FIG. 57 The structure of linker 9

FIG. 58 The HPLC profile of linker 9

FIG. 59 The ES I-MS of linker 9

### DETAILED DESCRIPTION

The present disclosure is further illustrated with the following specific examples, which, however, are not limitations to the present disclosure.

1. The preparation of linker 1

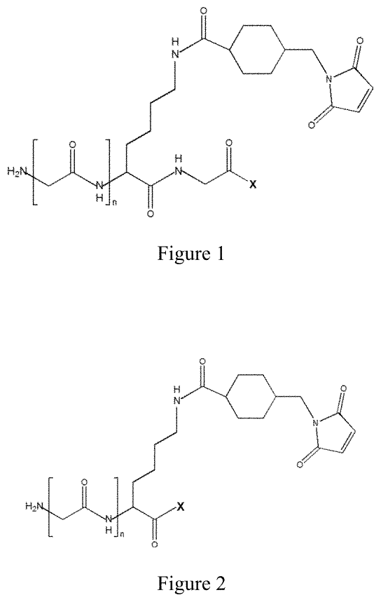

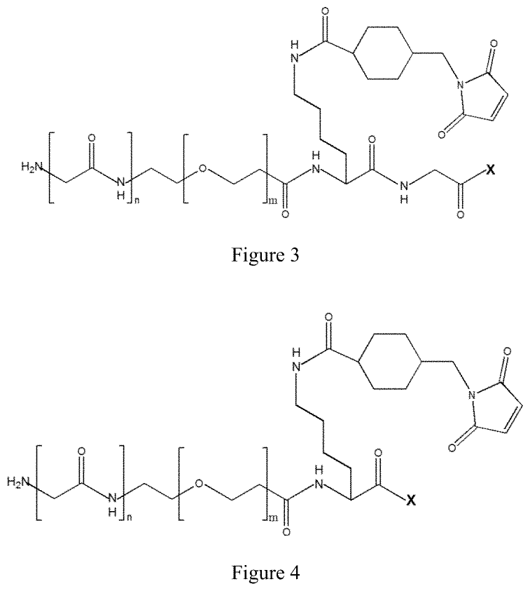

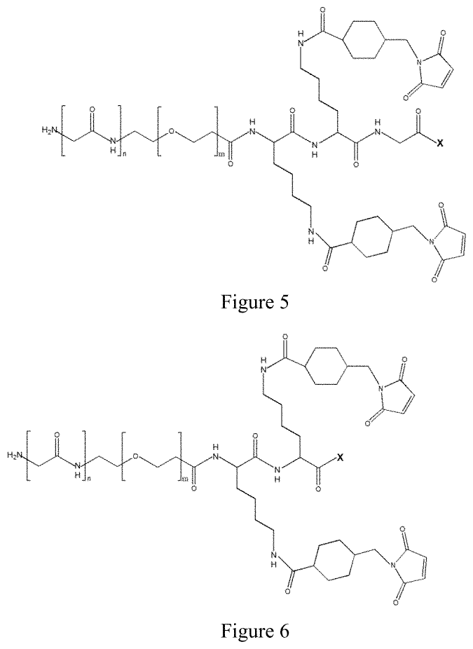

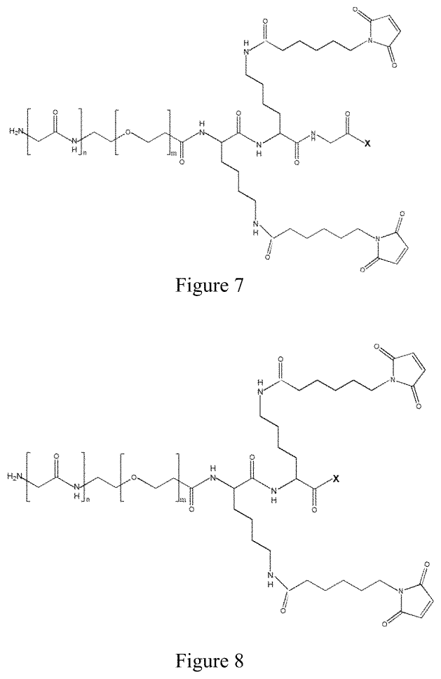

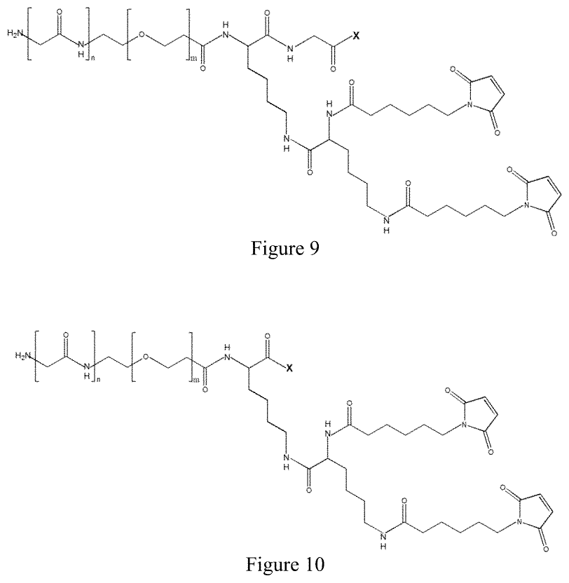

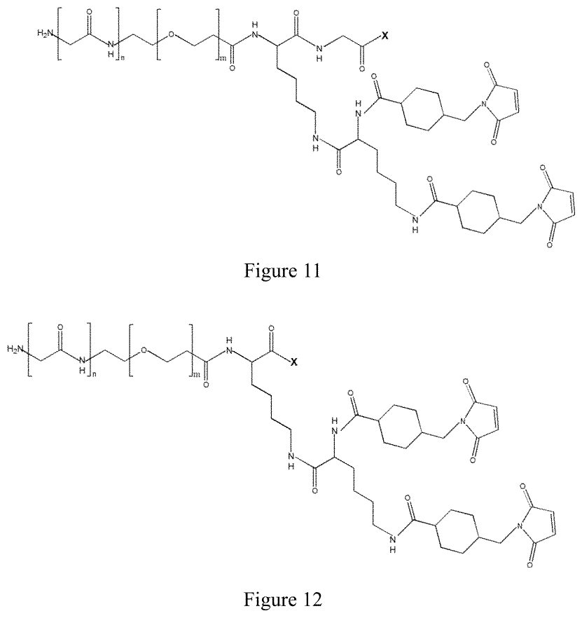

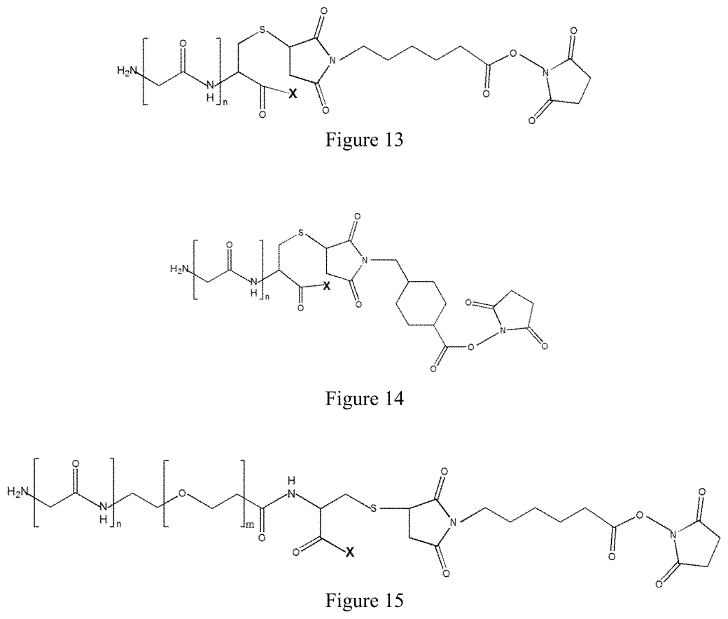

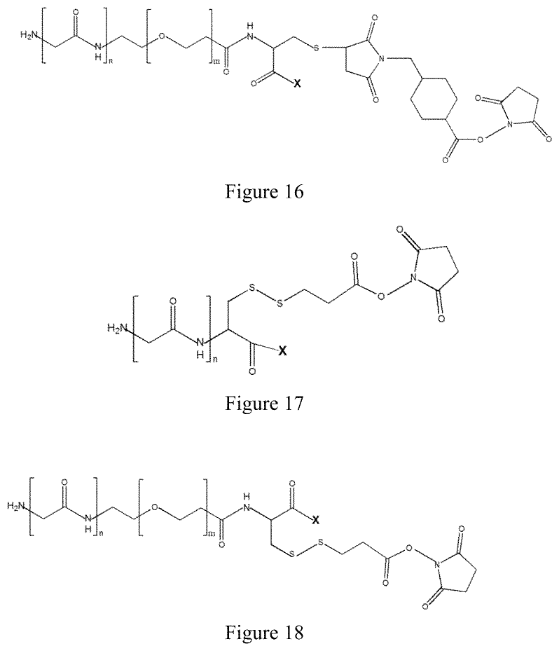

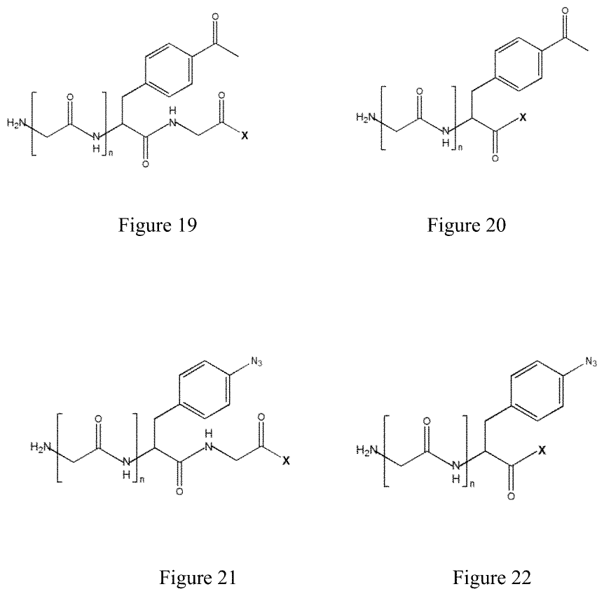

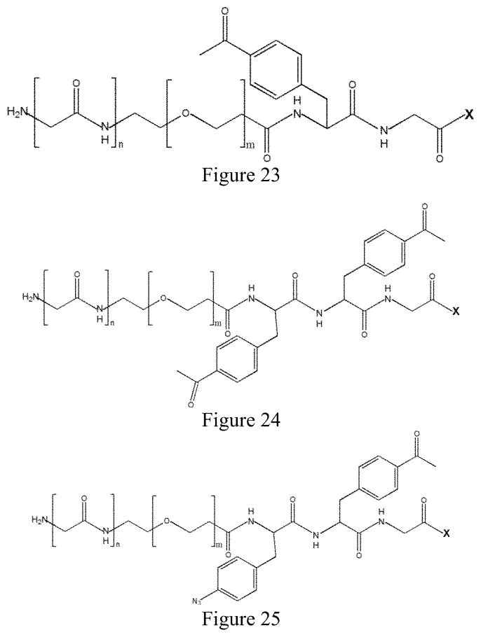

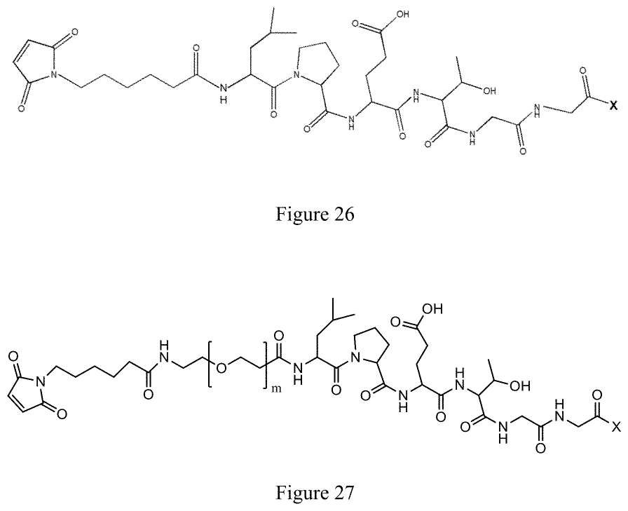

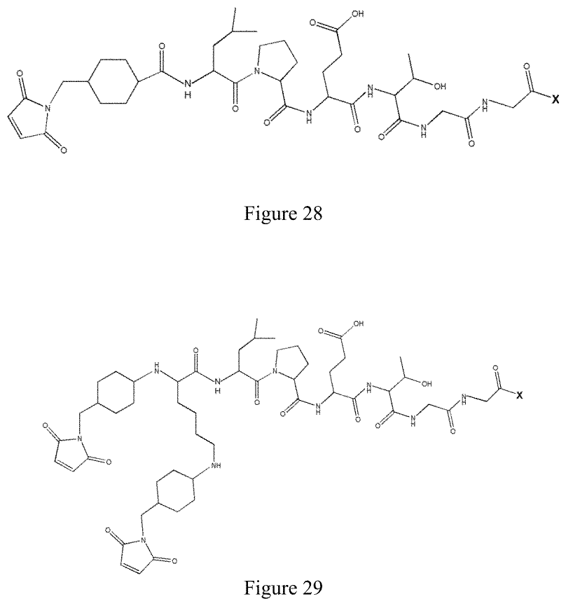

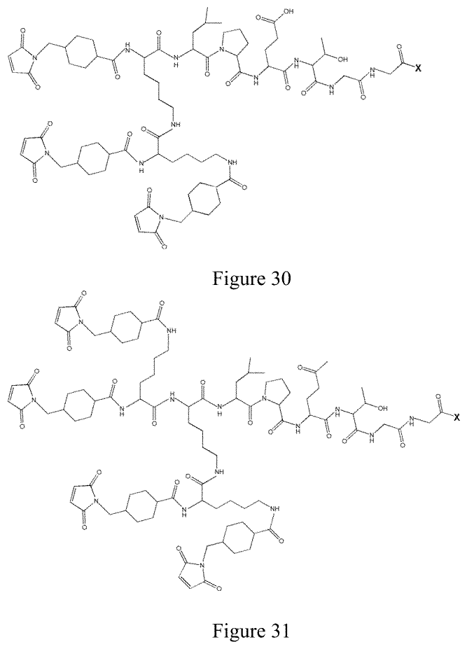

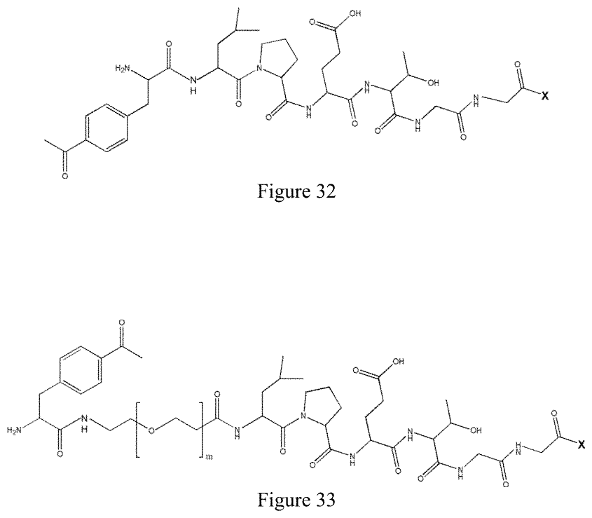

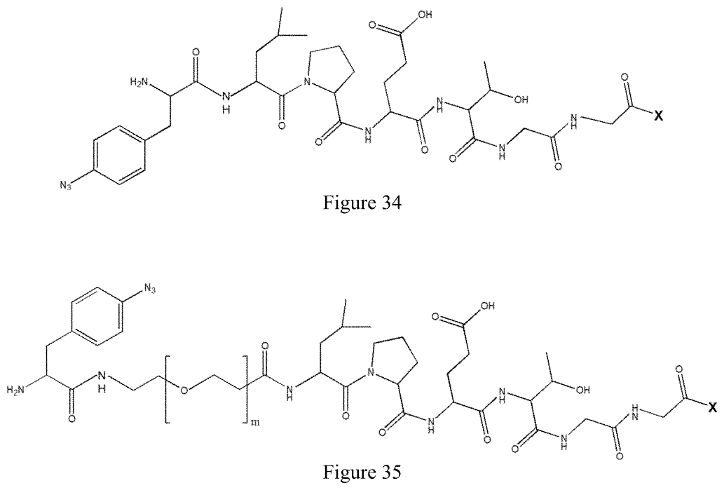

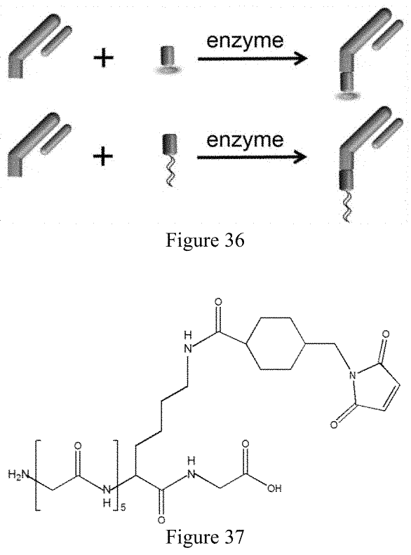

When n=5, X is —OH, the general formula of linker 1 shown in FIG. 1 is shown in

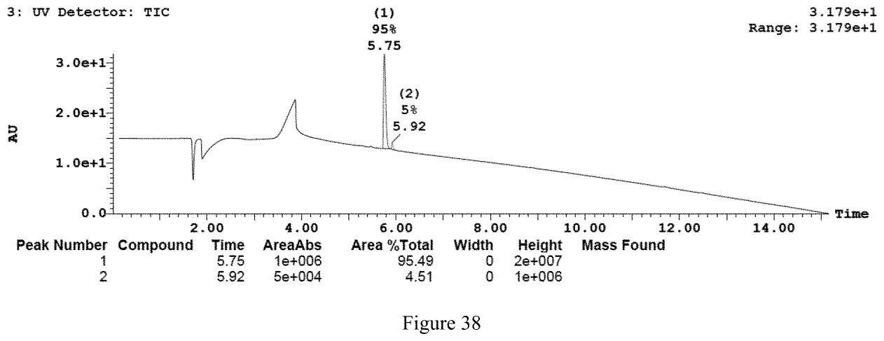

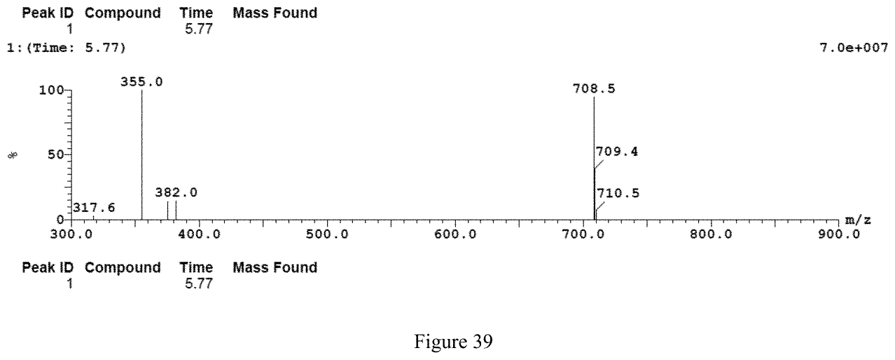

FIG. 37. The linker was prepared via solid phase peptide synthesis protocol on Wang resin using Fmoc Chemistry. The c-amino group of lysine was deprotected, and N-Succinim idyl 4-(N-maleimidomethyl) cyclohexane-1- carboxylate (SMCC) was chemically coupled to it in DMF. The linker was then cleaved from the resin and all protection groups were removed. The crude linker 1 was purified by HPLC, and characterized by ESI-MS. As shown in FIG. 38, the purity of the linker was 95.49%, and the found MS was 708.5 (M+1) shown in FIG. 39 (expected MW 707). This linker thus obtained will be coupled with small molecules, nucleic acids or tracer molecules.

2. The preparation of a coupling intermediate made of linker 1 and DM1

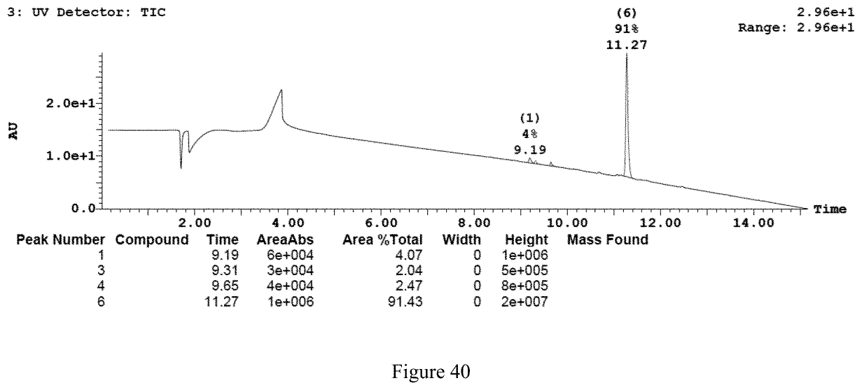

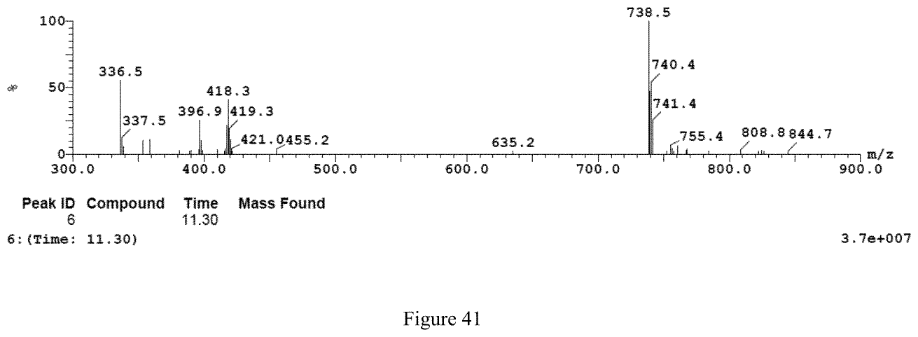

Maytansine derivative DM1 was purchased from Jiangyin Concortis Bio-Technology Co., Ltd. UPLC analysis showed a purity of 91.43% and ESI-MS showed a molecular weight of 738.5(expected 738). The results were shown in FIGS. 40 and 41.

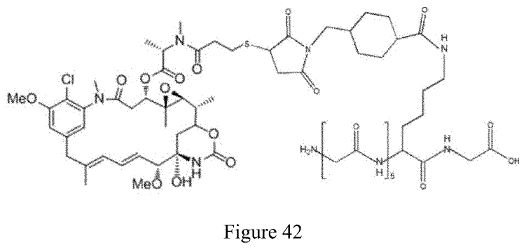

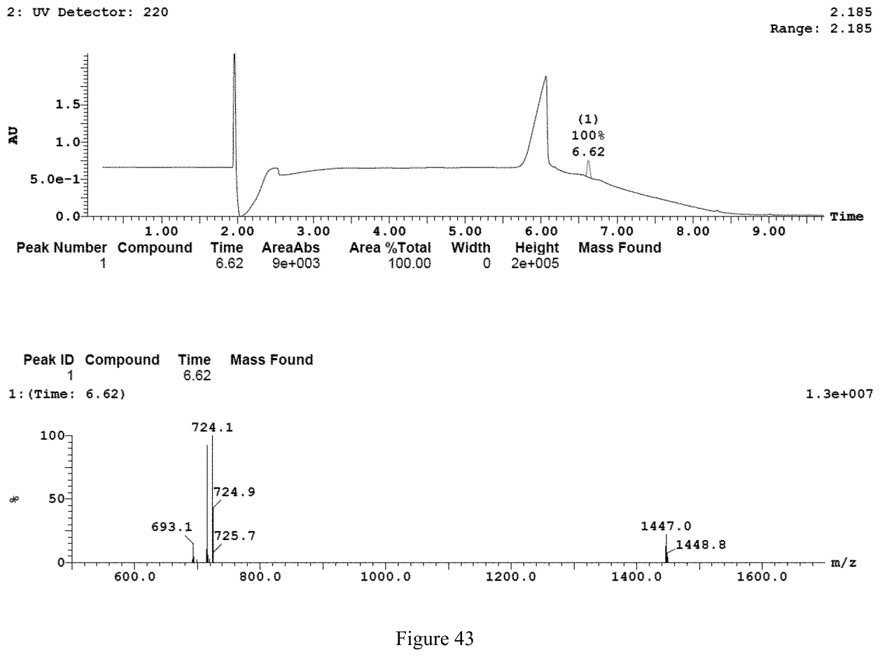

The synthetic linker 1 obtained above and the maytansine derivative DM1 were dissolved in a suitable solvent in equimolar ratio, the mixture was incubated at room temperature. The structure of the coupling intermediate is shown in FIG. 42. It was subjected to UPLC-MS analysis, and the results shown in FIG. 43. The coupling efficiency was 100%, expected molecular weight is 1447.9, ESI-MS found 1447(M-1).

The product obtained from the above procedure was site-specifically connected to a tumor-specific antibody or antibody analogue. The antibody-drug conjugate thus obtained was highly homogeneous, i.e., the number of drugs and the sites of coupling are highly specific. This highly homogenous ADC drugs can be used in a variety of tumor targeted therapies, including but not limited to breast cancer, stomach cancer, lung cancer, ovarian cancer and leukemia. In comparison with the ADCs already on the market, the highly homogenous new drugs prepared by the current invention, offer many advantages including but not limited to stability, reliability, efficacy and safety.

3. The preparation of linker 26

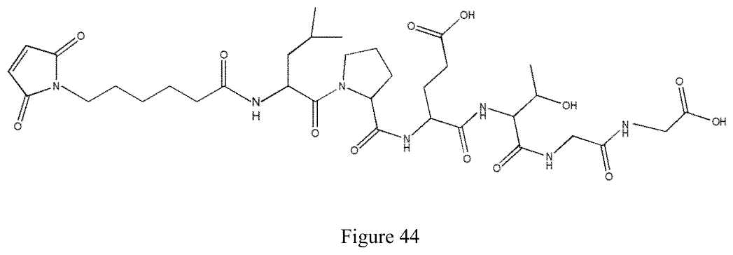

When X is —OH, the general linker structure shown in 26 becomes the structure shown in FIG. 44.

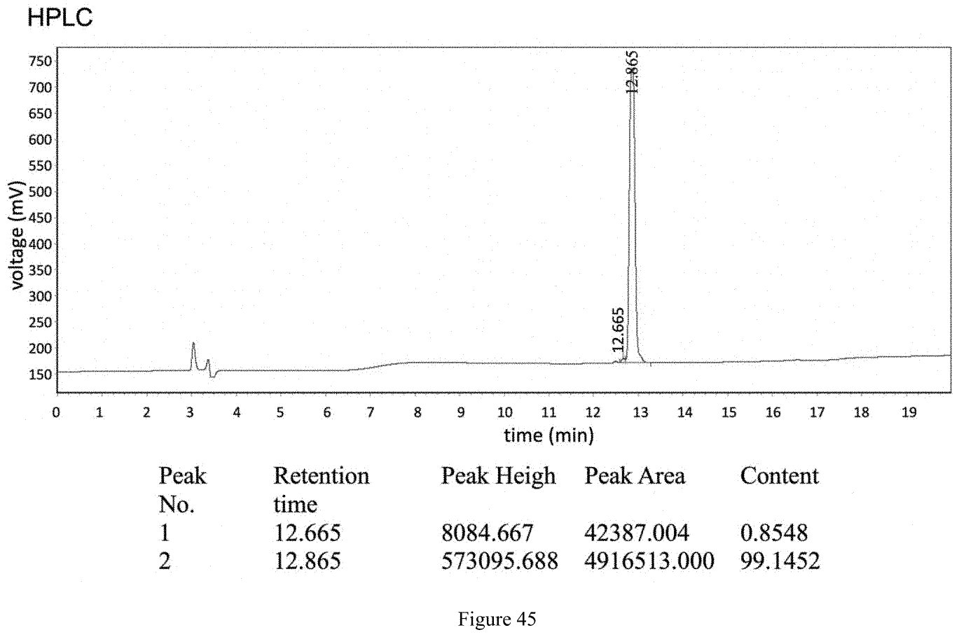

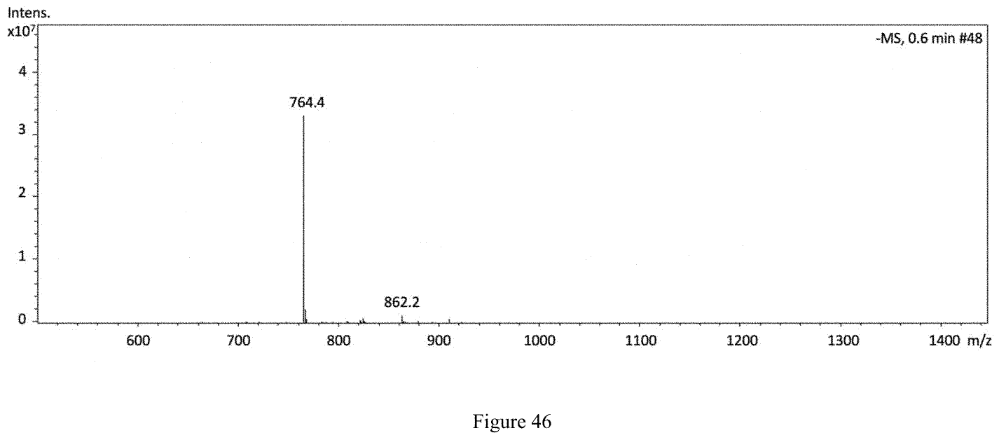

A similar method as used for the preparation of linker 1 was used. The crude product was purified by HPLC, characterized by ESI-MS analysis. As shown in FIG. 45, the purity of linker 26 was more than 99%; the expected molecular weight of 765, ESI-MS found 764 (M-1), as shown in FIG. 46.

The linker 26 and those alike may be used to react with small molecules, nucleic acids or tracer molecules.

4. The preparation of a conjugate intermediate with siRNA as the payload

A 5′-terminal thiol modified mice GAPDH siRNA was purchased from Genepharm Shanghai Ltd. The sequence of the said siRNA is:

5′-GUAUGACAACAGCCUCAAGdTdT-3′
3′-dTdTCAUACUGUUGUCGGAGUUC-5′.

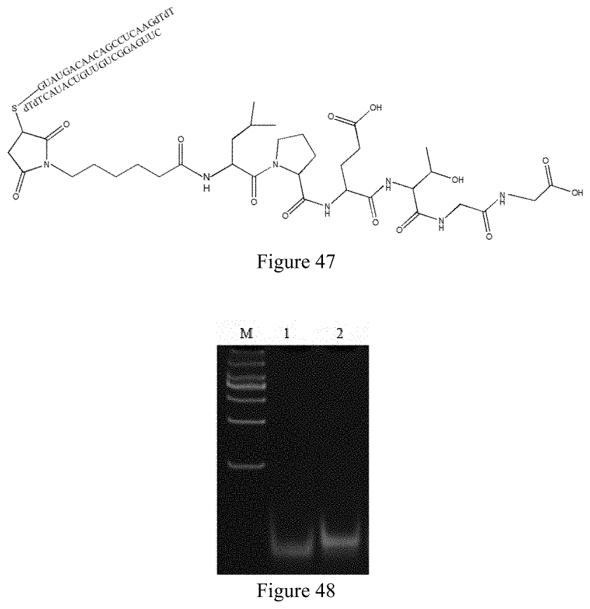

The modified siRNA and an excess of linker 26 were incubated in 1 x PBS buffer (pH7.4) at room temperature for 1-24 h. The extra linker 26 was removed by ultrafiltration to give a GAPDH siRNA-linker intermidiate as shown in FIG. 47. SDS PAGE indicated that the coupling efficency was >90% as shown in FIG. 48.

5. Enzyme catalysed site specific coupling of siRNA and Green Florecein Proten(GFP)

Recombinant GFP was purified by nickel affinity purification, treated with TEV enzyme to release the polyglycine sequence as the substrate for Sortase, and the resulted GGG-GFP protein was collected.

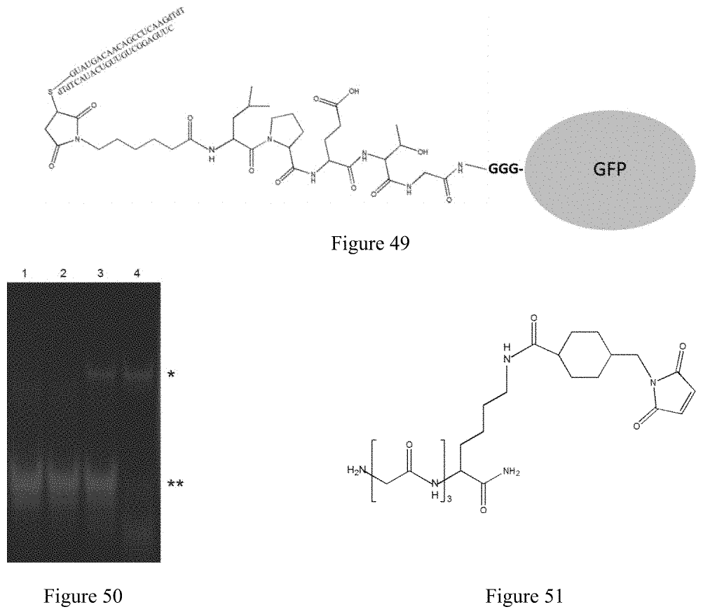

Excess amount of GAPDH siRNA linker intermediate 26 and GGG-GFP was site-specifically coupled by a genetically engineered Sortase A in 1× PBS buffer (containing Tris pH8.0, NaCI, CaCl2) at 37° C. for 2h. Samples were taken at different time intervals. The structure of the final product is shown in FIG. 49. 15% non-denaturing SDS PAGE showed that the coupling efficebcy was 80% in 2 h (FIG. 50).

This result clearly indicated that siRNA was site-specific coupling to a protein. An important application of this method is the site specific coupling of a tumor targeting antibody or antibody analogue with siRNA of therapeutic value, creating a new generation of targeting siRNA drugs. Another important application of this method is the coupling of tumor targeting antibody or antibody analogue with a tracer molecule which offers a new generation of tumor tracing agents.

6. The preparation of linkers 2, 3 and 9

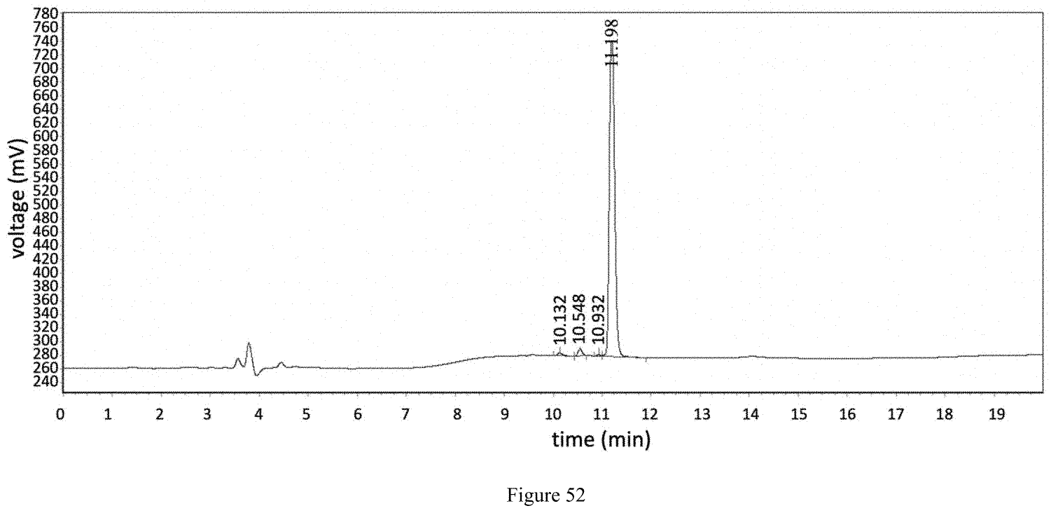

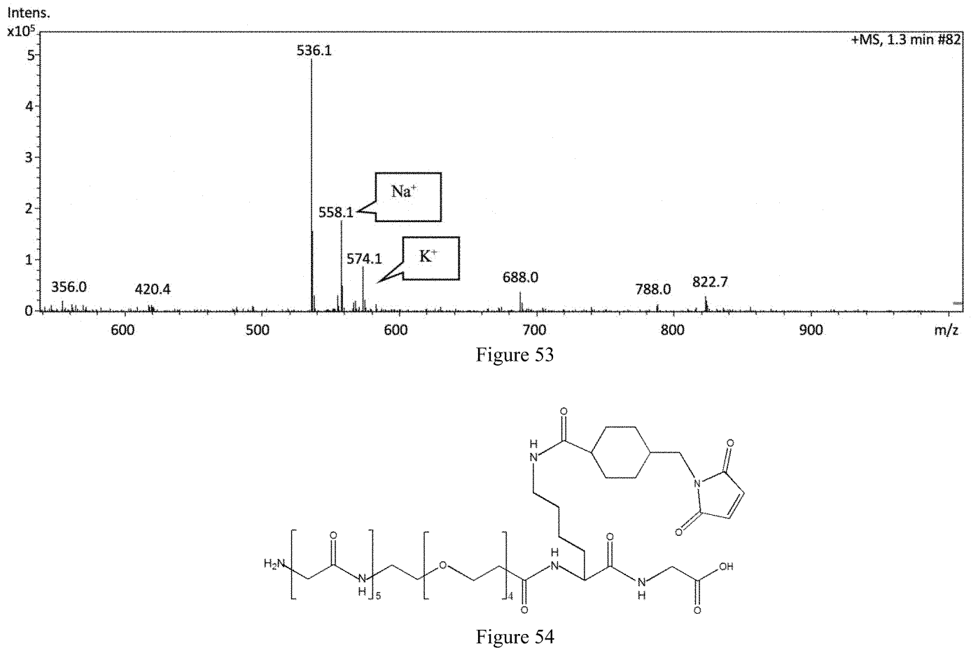

When n=3, X is —NH2, the structure in formulus 2 become linker 2 (FIG. 51). A similar method as described for linker 1 was used to prepare linker 2. After purification, it was analized by ESI-MS. As shown in FIGS. 52, the purity of linker 2 is 97.3492%. The expected MS of linker 2 is 535 and found 536 (M+1) (FIG. 53).

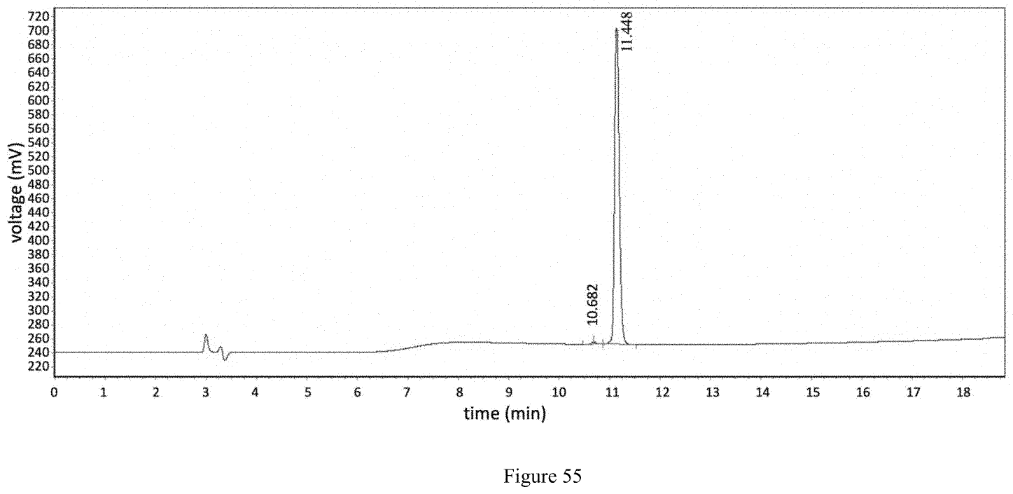

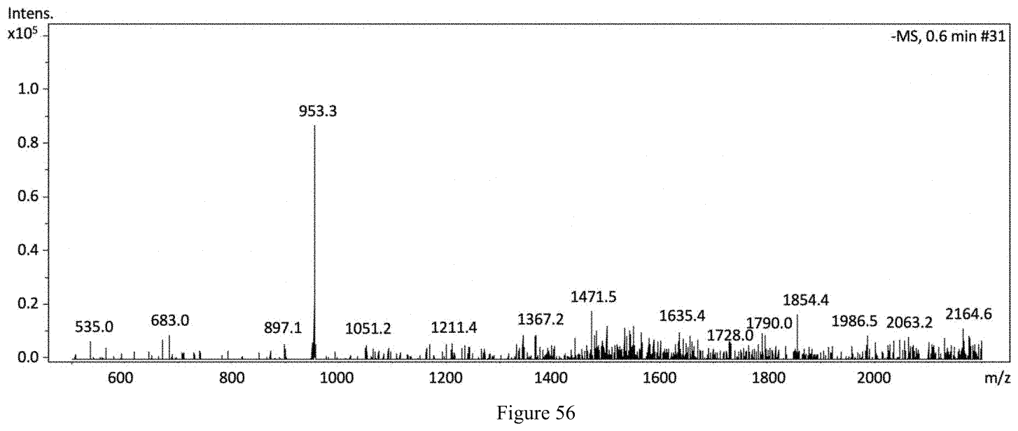

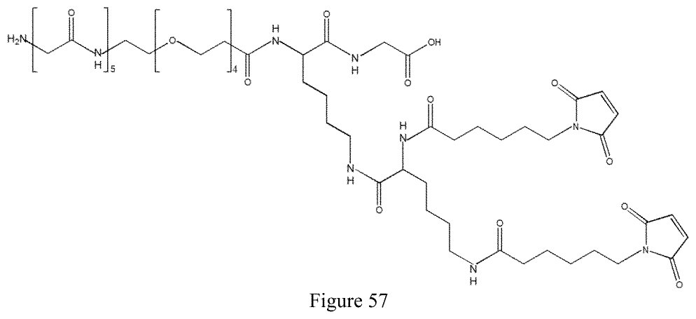

When n=5, m=4, X is —OH, the chemical structure of linker 3 was specified and shown in FIG. 54. Similar protocol as descrobed for linker 1 was applied with modification. The crude product was purified by HPLC. After purification, it was analized by ESI-MS. As shown in FIGS. 55, the purity of linker 3 is 99.3650%. The expected MS of linker 3 is 954 and found 953 (M +−1) (FIG. 56).

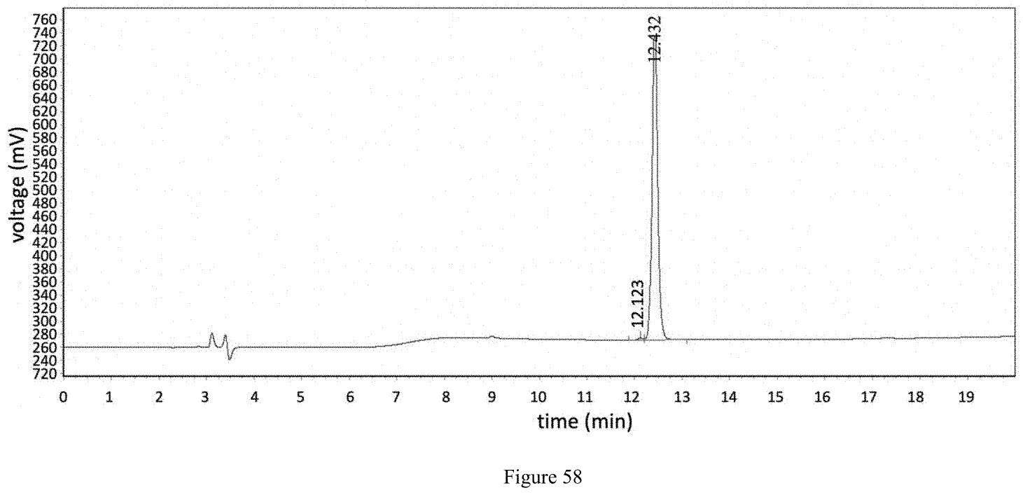

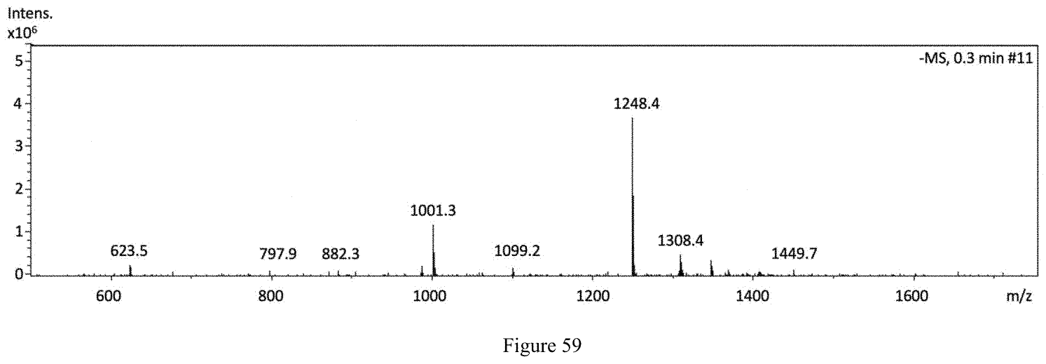

When n=5, m=4, X is —OH, the chemical structure of linker 9 was specified and shown in FIG. 57. Similar protocol as descrobed for linker 1 was applied with modification. The crude product was purified by HPLC. After purification, it was analized by ESI-MS. As shown in FIGS. 58, the purity of linker 3 is 99.3650%. The expected MS of linker 9 is 1249 and found 1248 (M-1) (FIG. 59).

Linkers 2, 3, 9 thus obtained can be used to couple with small molecules, nucleic acids, or tracer molecules. Linker 9 has two reactive functional groups which can react with two small molecules, nucleic acids or tracer molecules.

1.-60. (canceled)

61. A coupling intermediate having the structure of formula (Ill) or (IV):
PCA1−(LA)ₐ−CCA1−Payloadₕ (Ill),
or
Payloadₕ−CCA2−(LA)ₐ−PCA2 (IV),
wherein:
Payload is a nucleic acid;
h is an integer from 1 to 1000; when h>1, Payload is same or different;
PCA1 is a receptor substrate recognition sequence of Sortase;
PCA2 is a donor substrate recognition sequence of Sortase;
each of CCA1 and CCA2 is chemical conjugate region for connecting a payload to be connected;
LA is a connecting region, to connect PCA and CCA, wherein a is 0 or 1;
PCA1−(LA)ₐ−CCA1 (I) is selected from linkers 1-25 below; and
CCA2−(LA)ₐ−PCA2 (II) is selected from linkers 26-35 below: 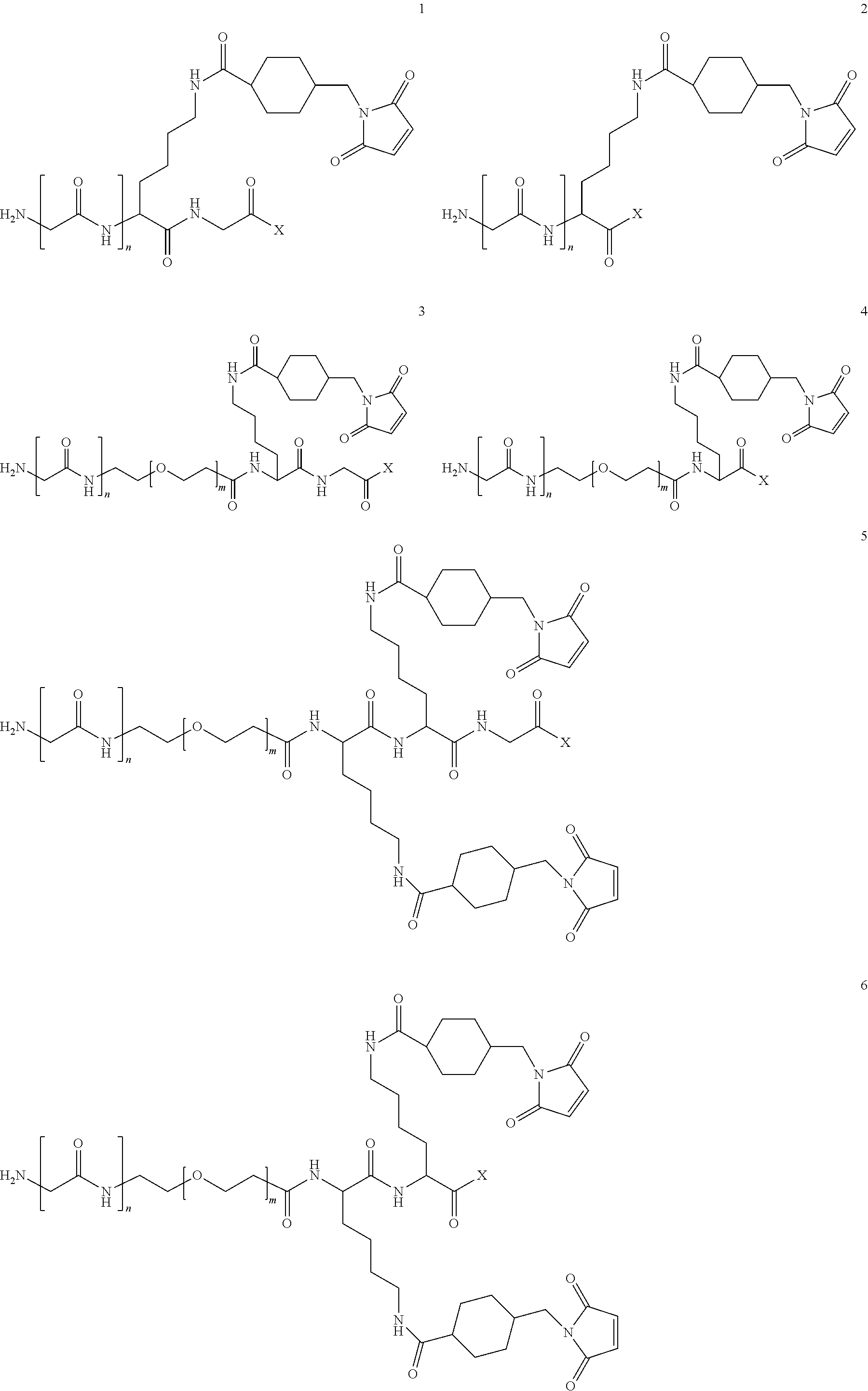 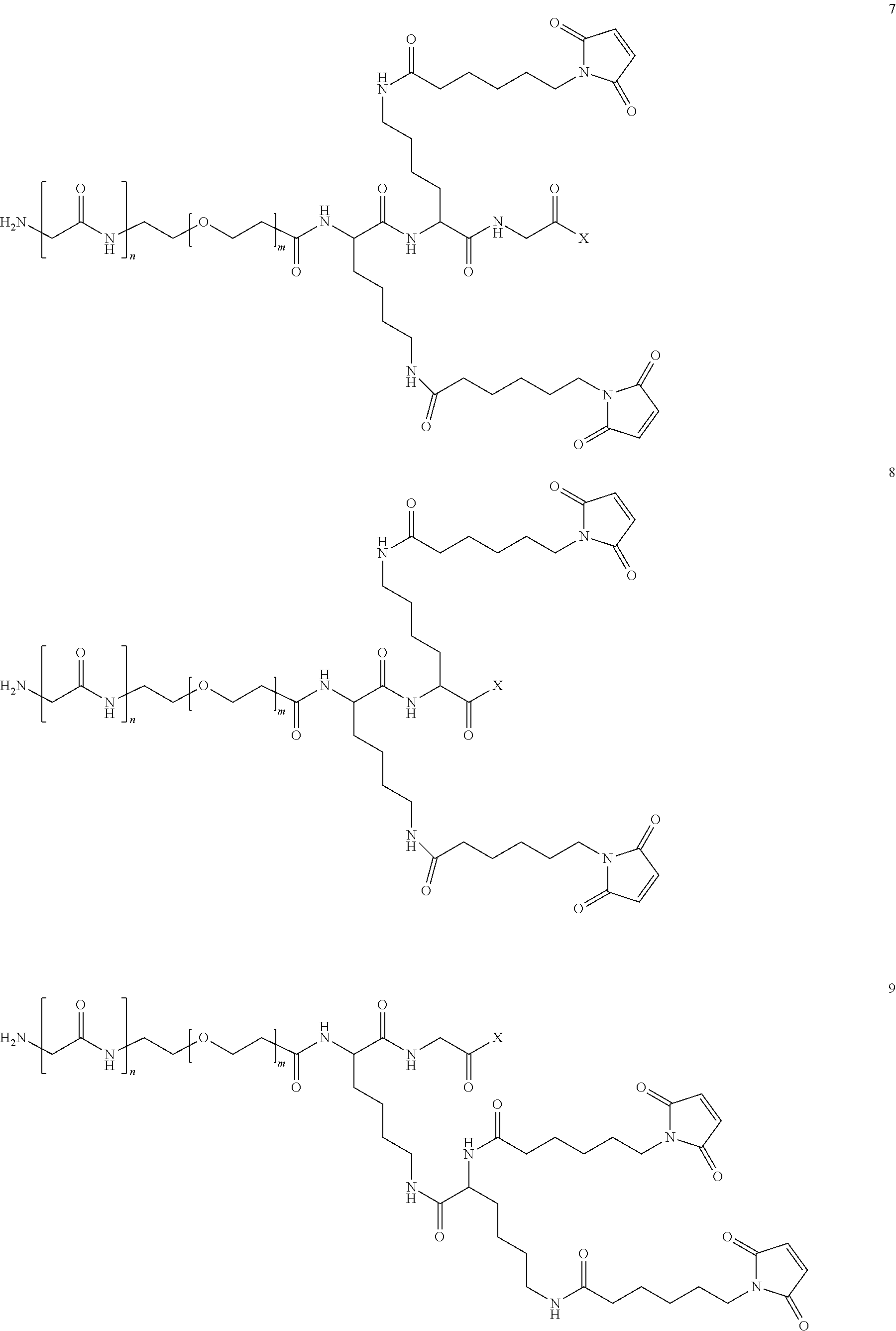 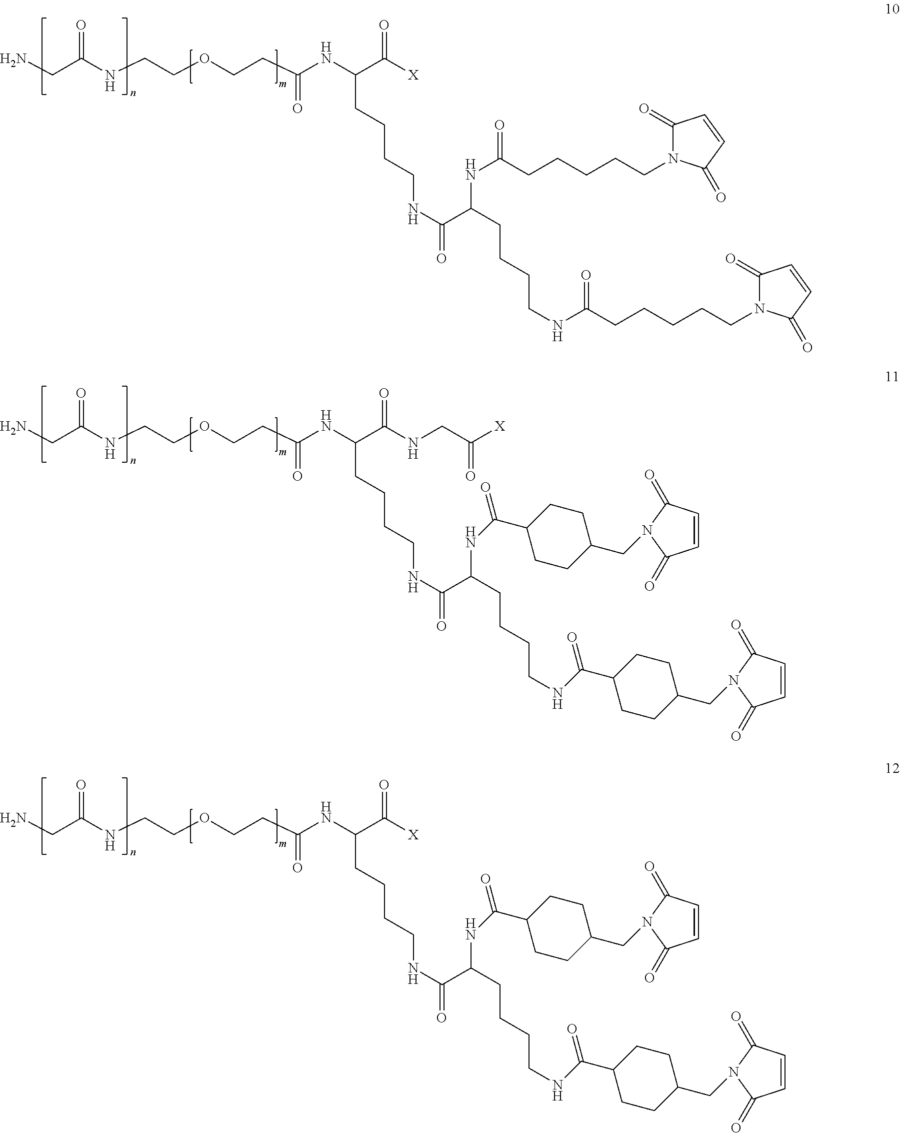 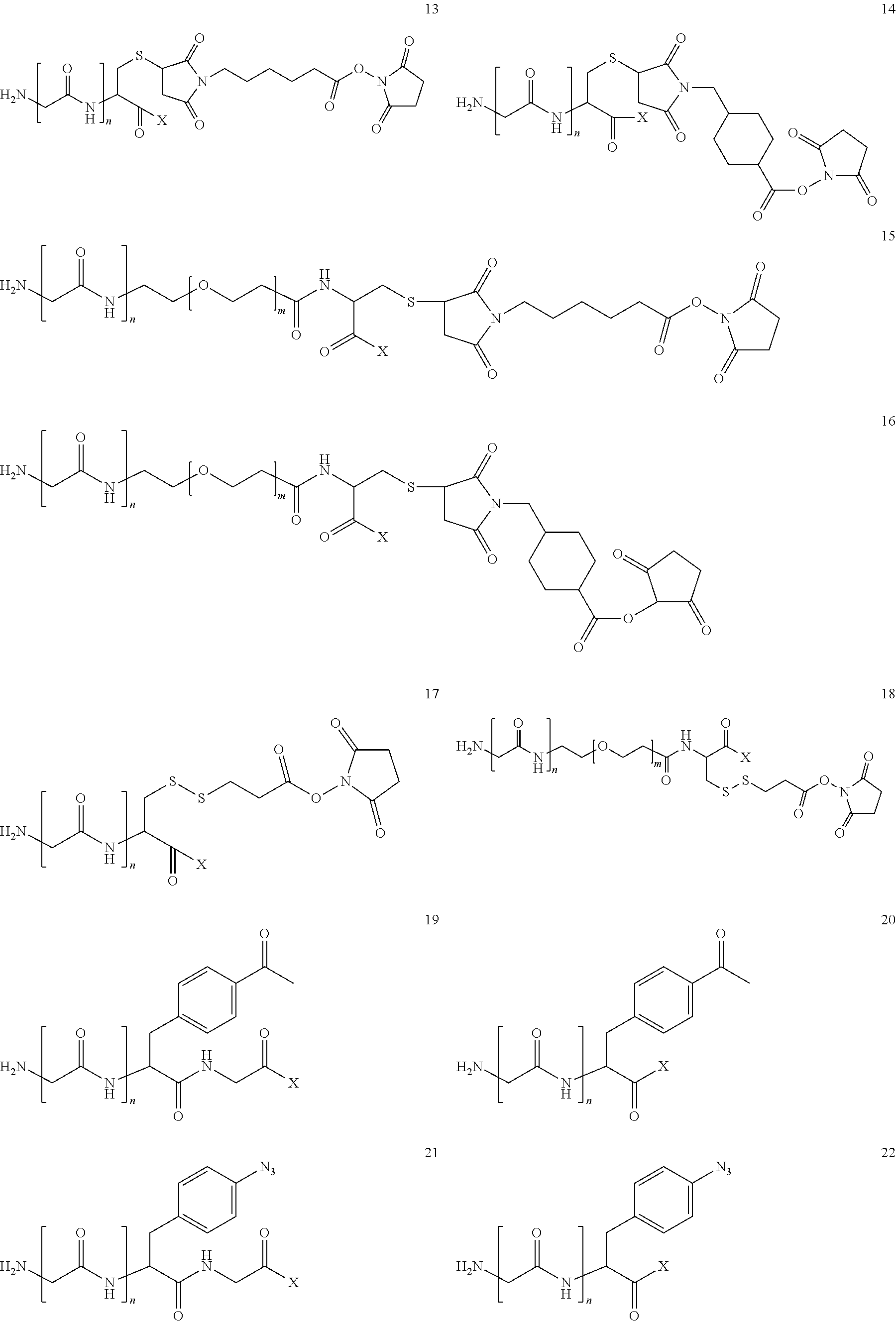 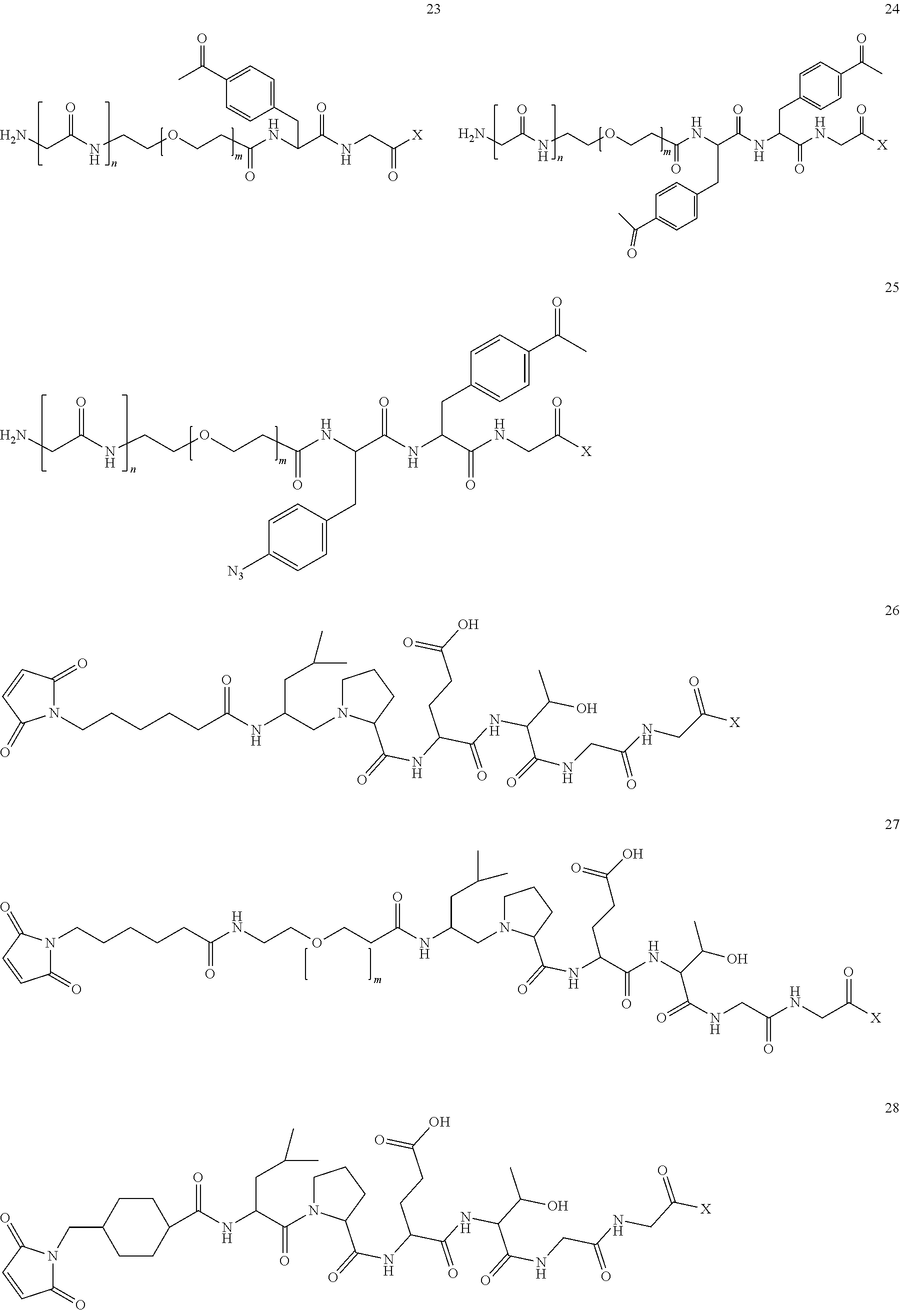 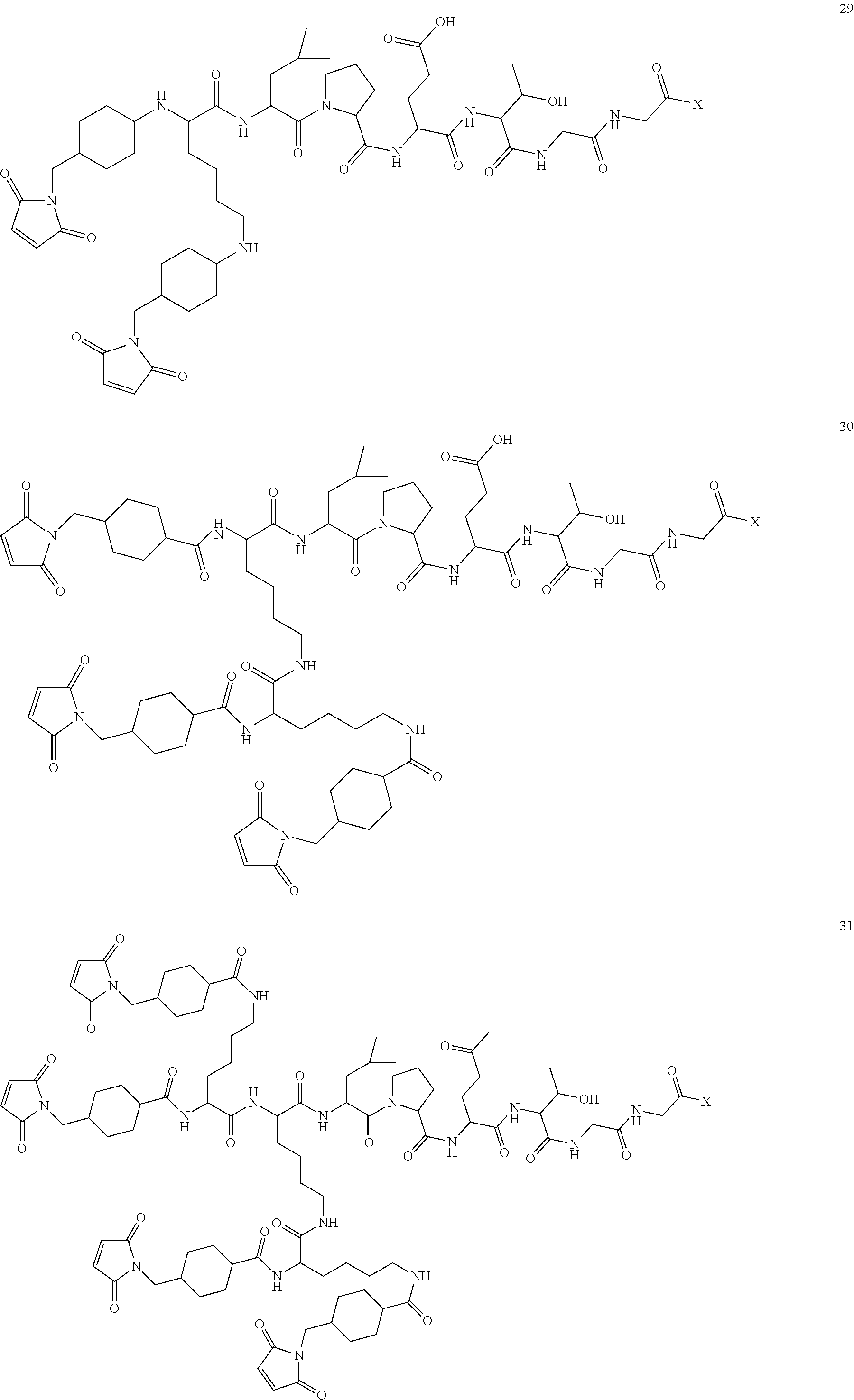 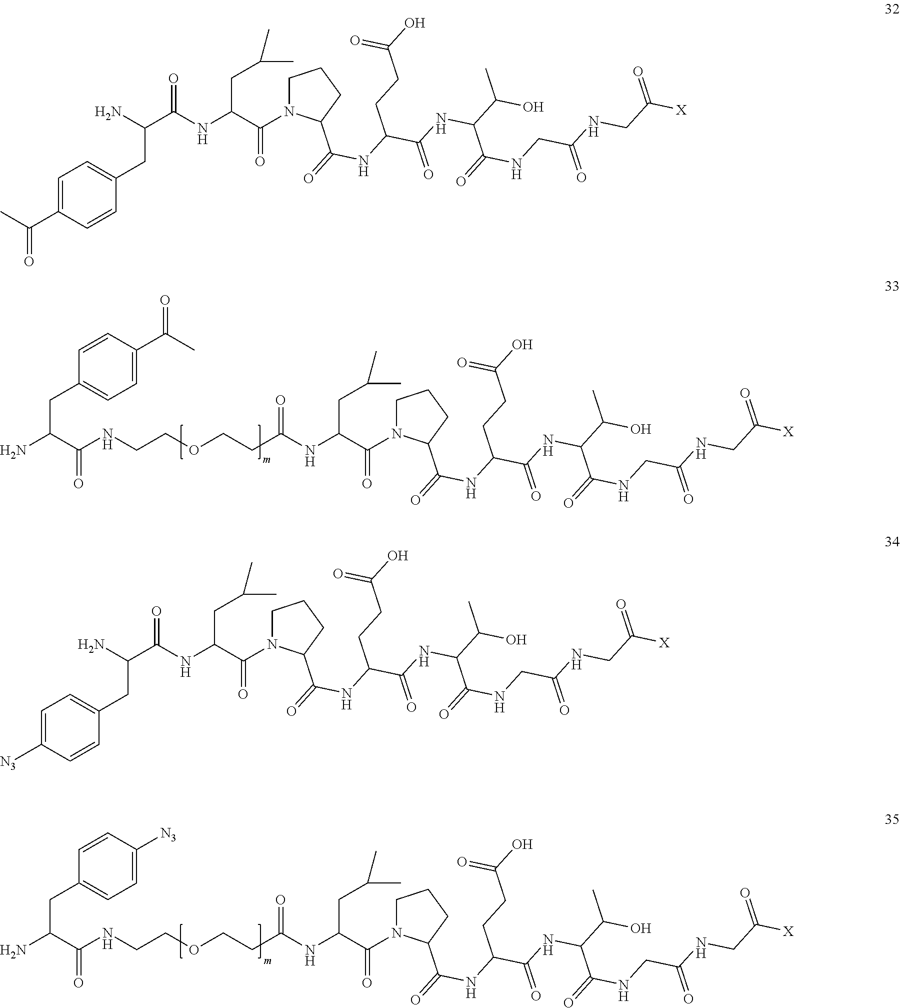
wherein n is an integer of 1-100, m is 0 or an integer 1-1000, and X is −OH or −NH₂.

62. The coupling intermediate according to claim 61, wherein CCA2−(LA)ₐ−PCA2 (II) is linker 26: 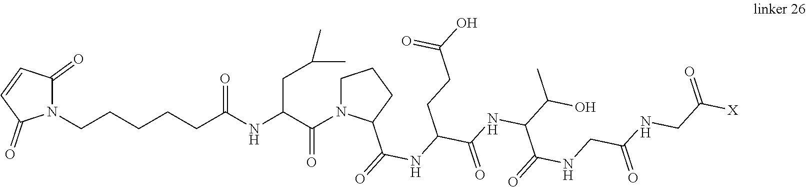
linker 26 and wherein n is an integer of 1-100, m is 0 or an integer 1-1000, and X is —OH or —NH₂.

63. The coupling intermediate according to claim 61, wherein X is —OH.

64. The coupling intermediate according to claim 61, wherein the nucleic acid is a siRNA.

65. The coupling intermediate according to claim 64, wherein the sequence of the siRNA is:
5′-GUAUGACAACAGCCUCAAGdTdT-3′
3′-dTdTCAUACUGUUGUCGGAGUUC-5′.

66. The coupling intermediate according to claim 61, wherein in preparation of the nucleic acid, a modification group of thiol, hydroxyl, carboxyl, amino, alkoxy-amino, alkynyl, azide or tetrazine is introduced at a preferred position, in order to covalently link with PCA1−(LA)ₐ−CCA1 (I) or CCA2−(LA)ₐ−PCA2 (II).

67. The coupling intermediate according to claim 66, the modification group is thiol.

68. The coupling intermediate according to claim 61, wherein the coupling intermediate has the structure of the following formula 47:

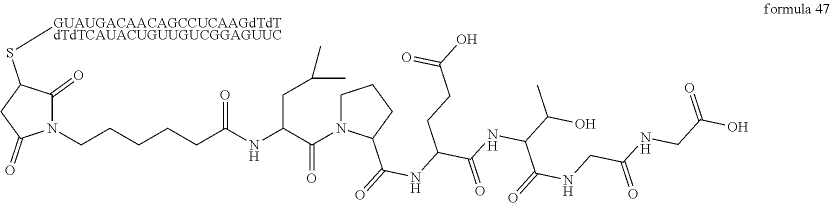

69. A targeting drug conjugate, wherein the said
conjugate having a structure represented by the formula (V) or (VI):
T−PCA1−(LA)ₐ−CCA1−Payloadₕ (V) or
Payloadₕ−CCA2−(LA)ₐ−PCA2−T (VI)
wherein:
Payload is a nucleic acid;
T is a targeting moiety;
h is an integer from 1 to 1000, when h>1, Payload is same or different; and
h is an integer from 1 to 1000; when h>1, Payload is same or different;
PCA1 is a receptor substrate recognition sequence of Sortase;
PCA2 is a donor substrate recognition sequence of Sortase;
each of CCA1 and CCA2 is chemical conjugate region for connecting a payload to be connected;
LA is a connecting region, to connect PCA and CCA, wherein a is 0 or 1;
PCA1−(LA)ₐ−CCA1 (I) is selected from linkers 1-25 below; and
CCA2−(LA)ₐ−PCA2 (II) is selected from linkers 26-35 below: 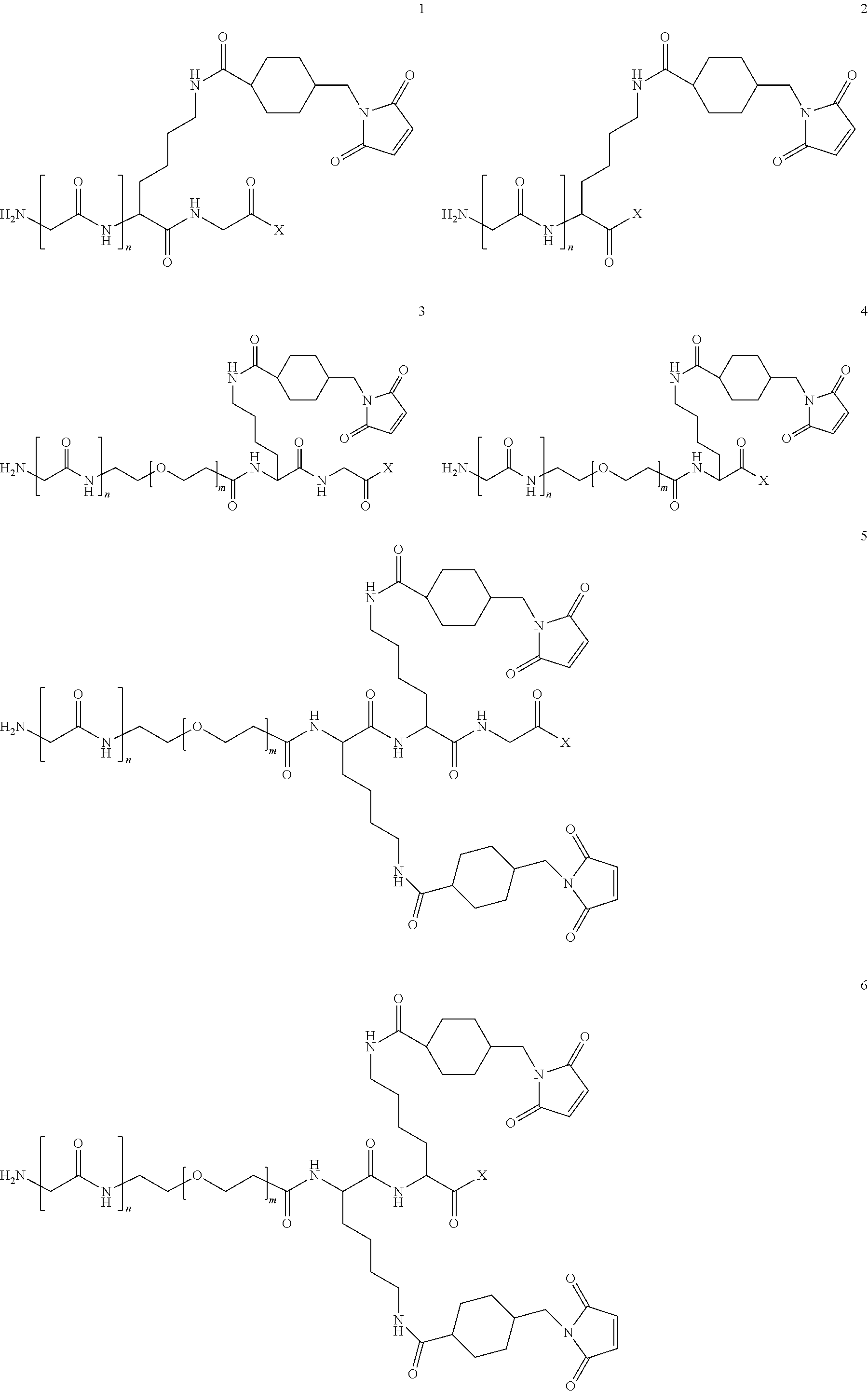 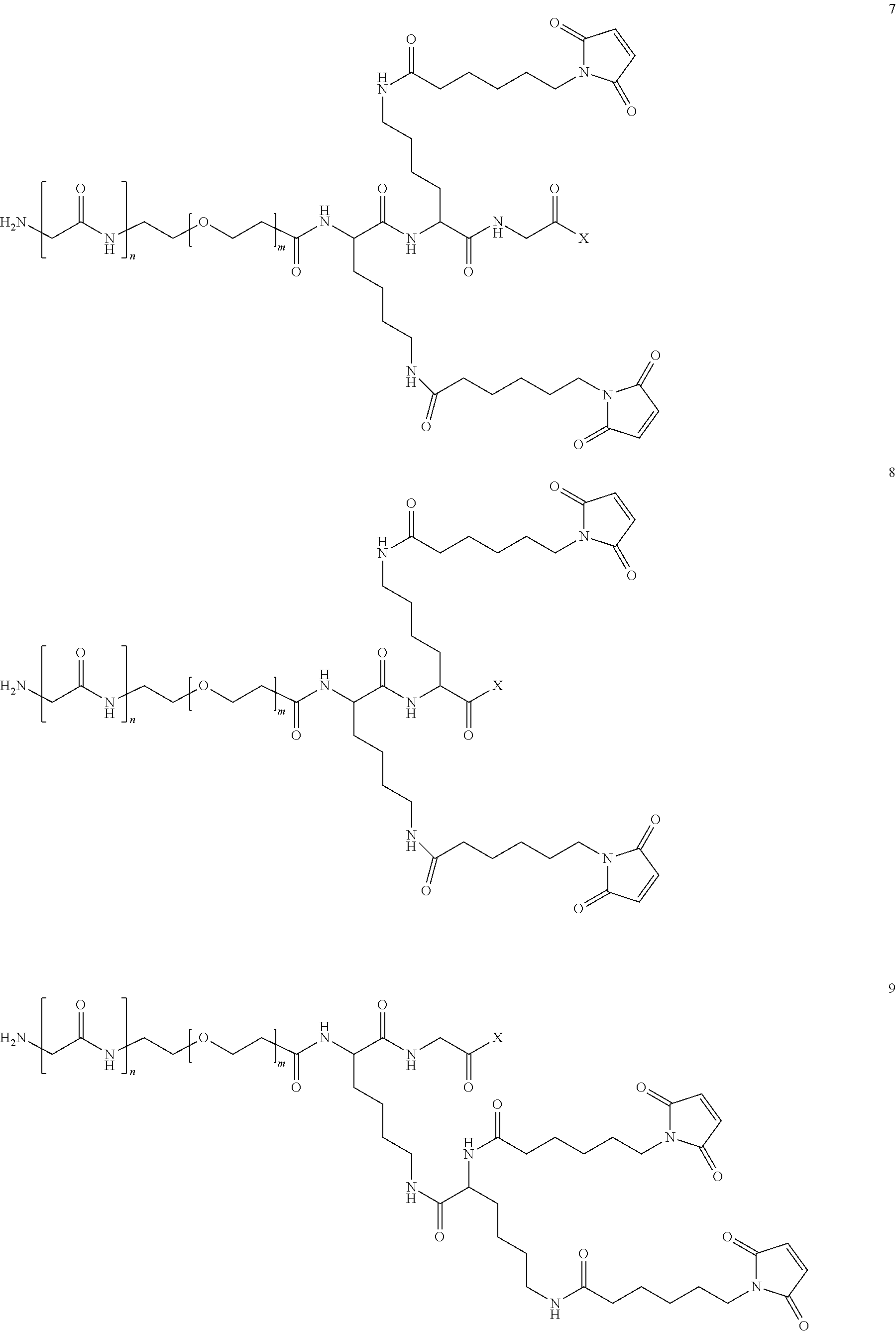 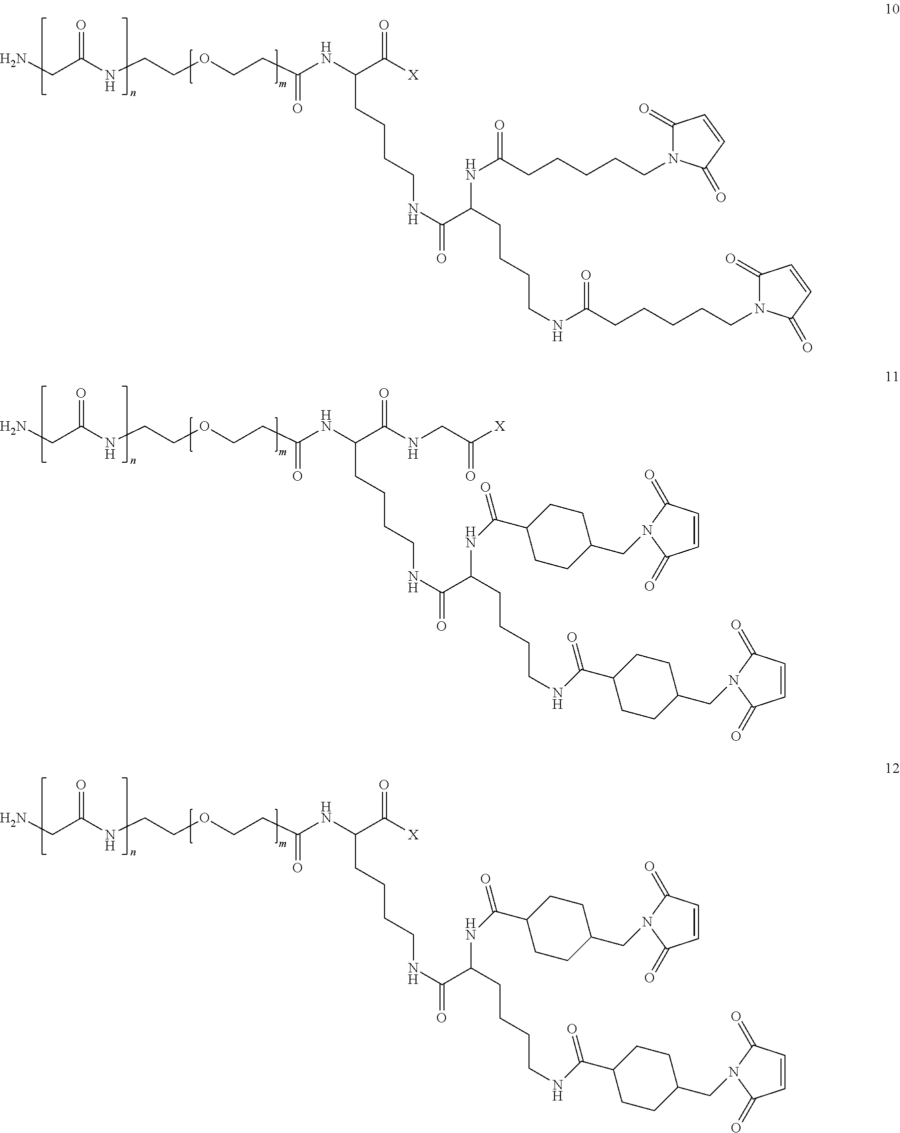 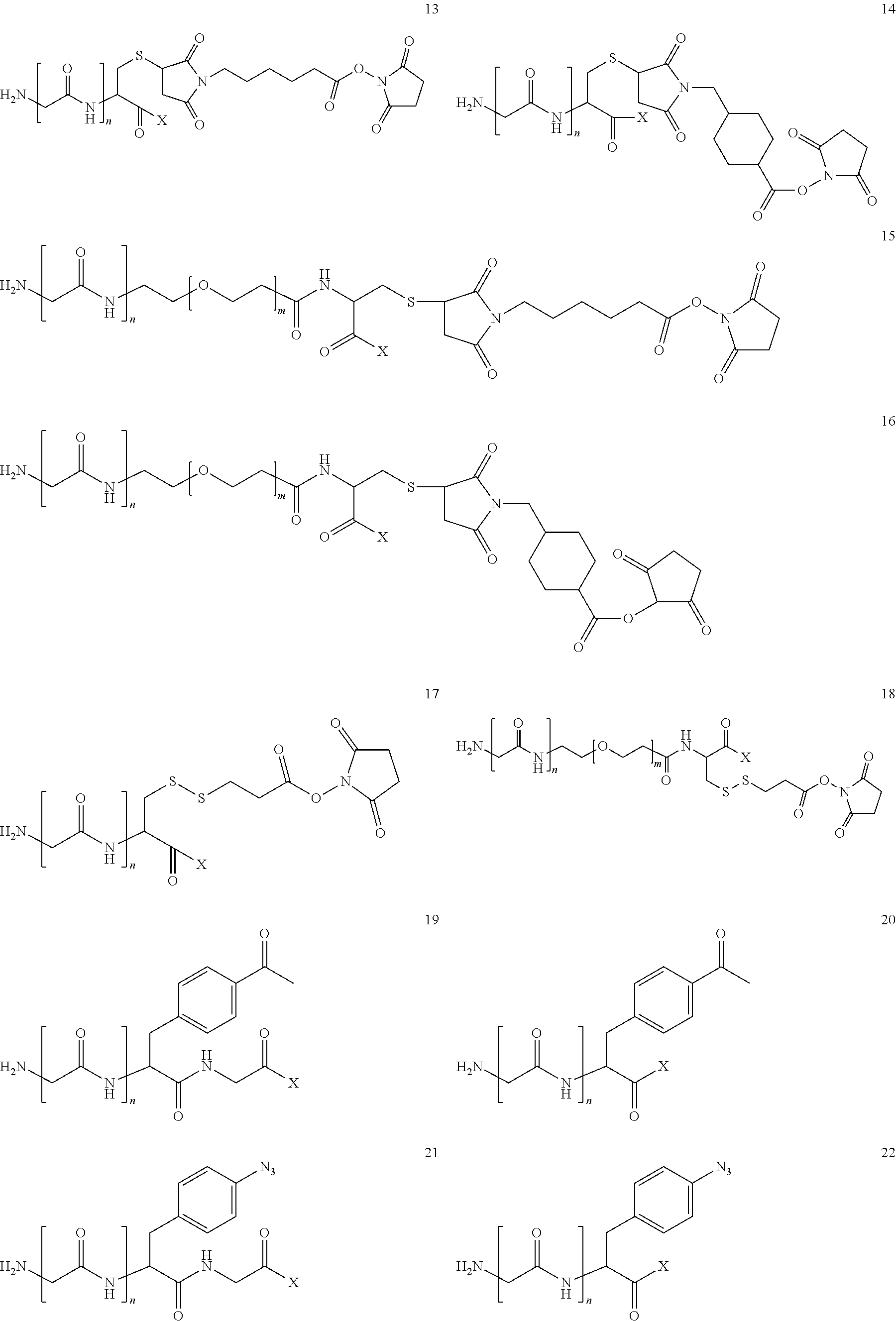 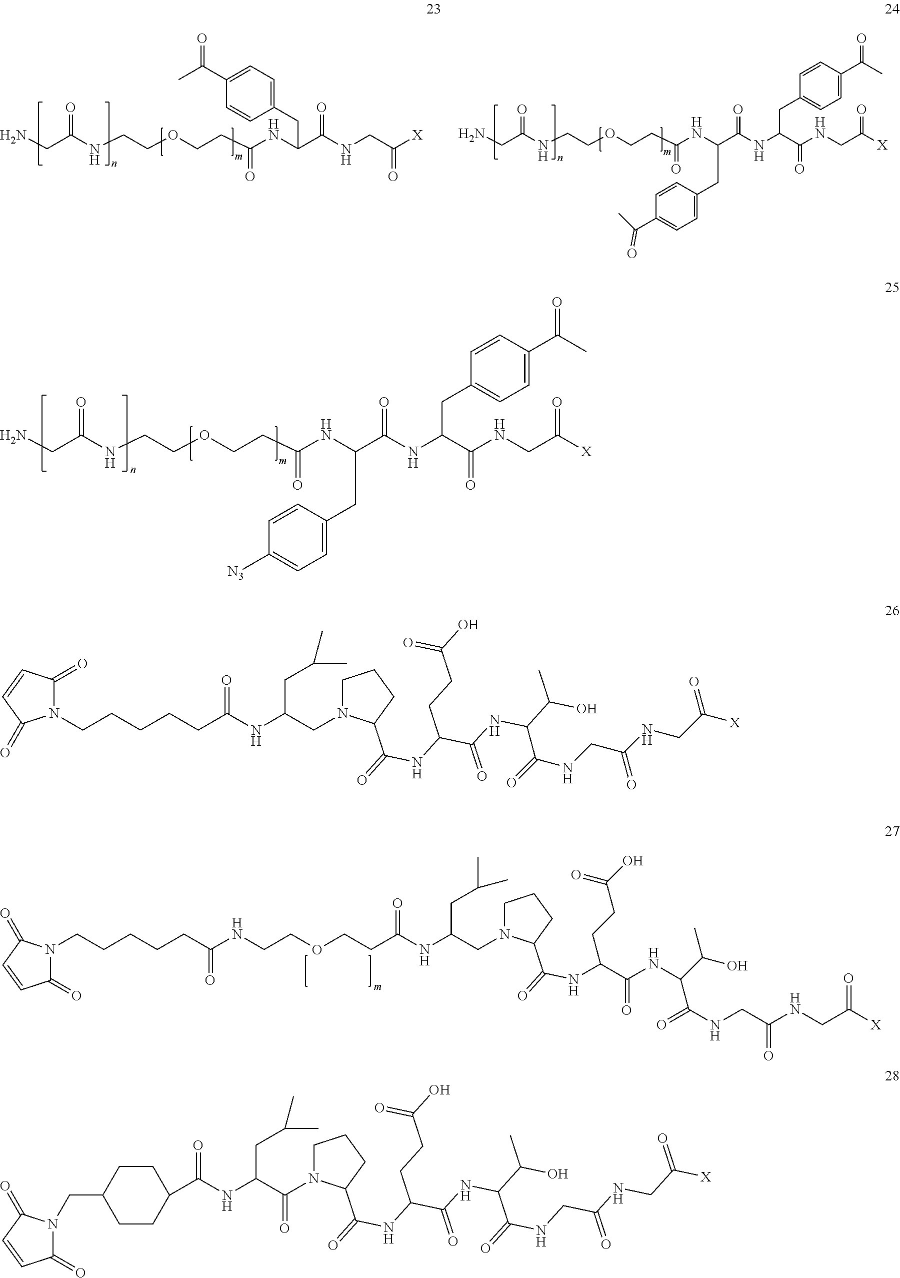 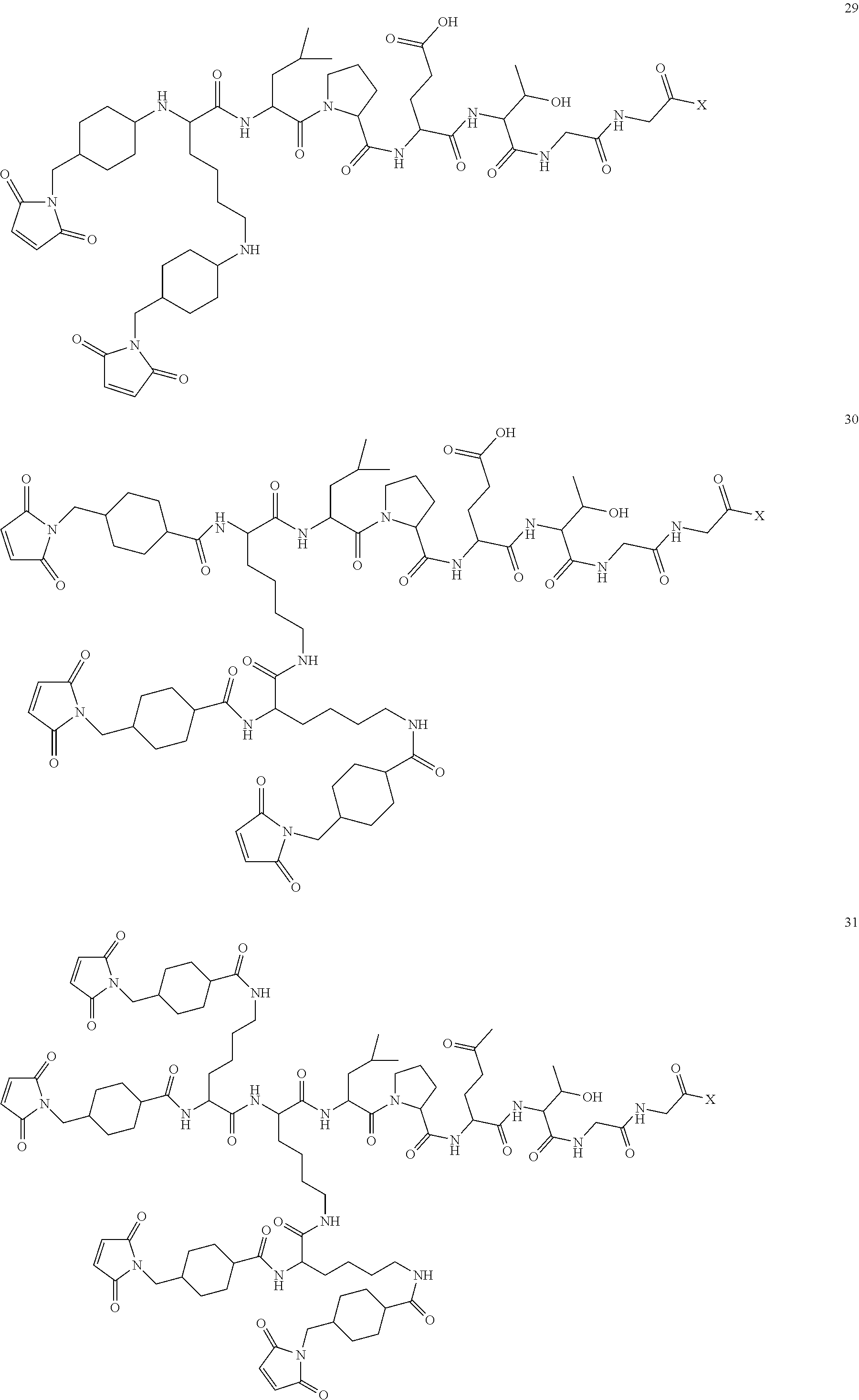 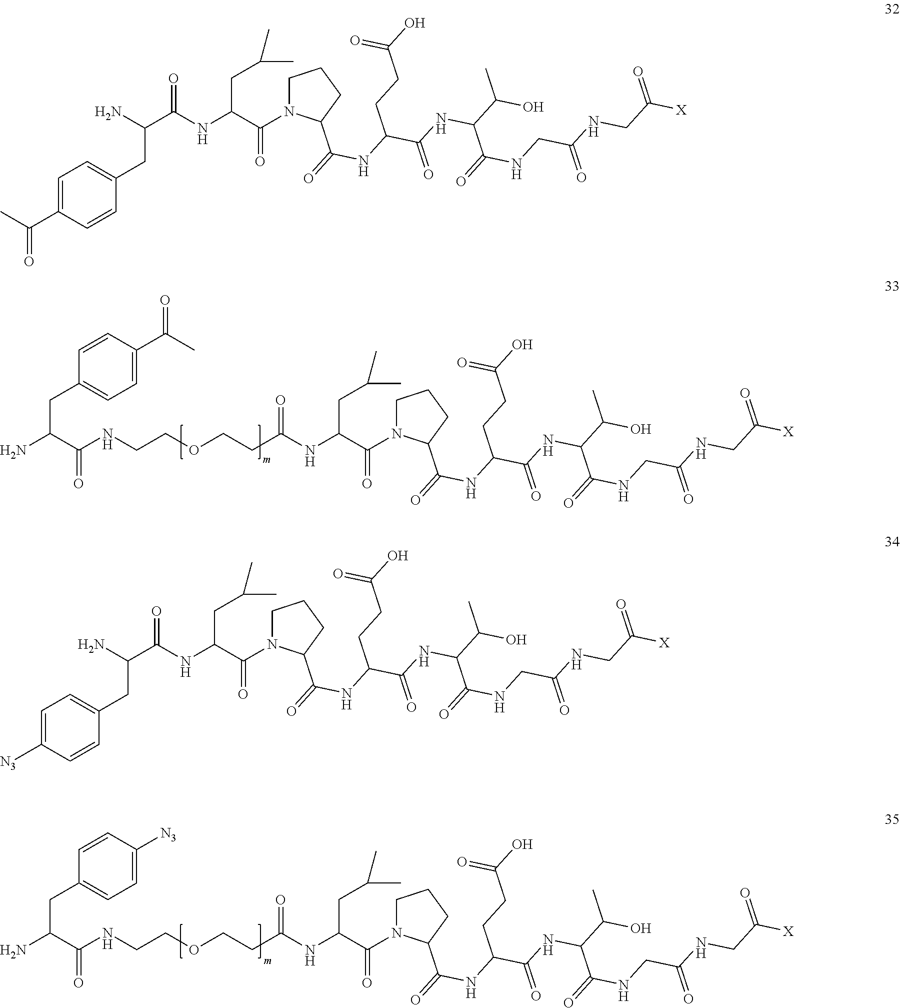
wherein n is an integer of 1-100, m is 0 or an integer 1-1000, and X is —OH or —NH₂, and wherein the PCA1−(LA)ₐ−CCA1 (I) or CCA2−(LA)ₐ−PCA2 (II) is site-specifically coupled to the targeting moiety.

70. The coupling intermediate according to claim 69, wherein CCA2−(LA)ₐ−PCA2 (II) is linker 26: 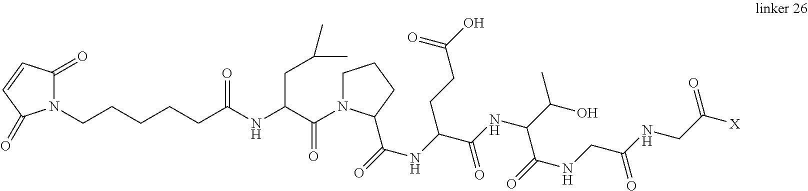
wherein X is —OH or —NH₂, and wherein the linker 26 is site-specifically coupled to the targeting moiety.

71. The targeting drug conjugate according to claim 69, wherein the nucleic acid is a siRNA.

72. The targeting drug conjugate according to claim 71, wherein the sequence of the siRNA is:
5′-GUAUGACAACAGCCUCAAGdTdT-3′
3′-dTdTCAUACUGUUGUCGGAGUUC-5′.

73. The targeting drug conjugate according to claim 69, wherein in preparation of the nucleic acid, a modification group of thiol, hydroxyl, carboxyl, amino, alkoxy-amino, alkynyl, azide or tetrazine is introduced at a preferred position, in order to covalently link with PCA1-(LA)ₐ−CCA1 (I) or CCA2−(LA)ₐ−PCA2 (II).

74. The targeting drug conjugate according to claim 73, wherein the modification group is thiol.

75. The targeting drug conjugate according to claim 69, wherein the targeting moiety is an antibody, a single chain antibody, a nano-antibody, a single domain antibody, an antibody fragment, an antibody mimetics, or a cell specific binding protein/peptide.

76. The targeting drug conjugate according to claim 69, wherein the targeting moiety is capable of binding to a target cell of: a tumor cell, a commonly used genetic engineering transfected cell, a virus-infected cell, a microorganism infected cell or a primary cultured cell.

77. A pharmaceutical composition, wherein the said composition comprises the targeting drug conjugate according to claim 69 and a pharmaceutically acceptable carrier or excipient.

78. A method for treatment of a disease of a subject comprising administration of the pharmaceutical composition according to claim 77 in an effective amount to the subject.

79. The method for treatment of a disease of a subject according to claim 78, wherein the said disease is selected from cancers, autoimmune diseases, inflammatory diseases, cardiovascular diseases and neurodegenerative diseases.

80. A method for preparing the targeting drug conjugate according to claim 69, which comprises connecting the coupling intermediate according to claim 61, with a targeting moiety (T) in a site specific way with Sortase.


SEQUENCE LISTING (9 sequences)

SEQ ID NO: 1 - protein, 5 aa, Staphylococcus aureus
  MISC_FEATURE (3)..(3): Wherein Xaa represents any amino acid
LPXTG

SEQ ID NO: 2 - protein, 5 aa, Staphylococcus aureus
NPQTN

SEQ ID NO: 3 - protein, 5 aa, Bacillus anthracis
NPKTG

SEQ ID NO: 4 - protein, 5 aa, Streptococcus pyogenes
  MISC_FEATURE (3)..(3): Wherein Xaa represents any amino acid
LPXTG

SEQ ID NO: 5 - protein, 5 aa, Streptomyces coelicolor
  MISC_FEATURE (3)..(3): Wherein Xaa represents any amino acid
LAXTG

SEQ ID NO: 6 - protein, 7 aa, Lactobacillus plantarum
LPQTSEQ

SEQ ID NO: 7 - protein, 6 aa, Artificial Sequence
  an artificial protein sequence for a Protein Conjugation Area (PCA)
  MISC_FEATURE (1)..(6): Xaa1 represents leucine (Leu) or asparagine (Asn), Xaa2 represents proline (Pro) or alanine (Ala), Xaa3 represents any amino acid, Xaa4 is threonine (Thr), Xaa5 represents glycine (Gly), serine (Ser), or asparagine (Asn), Xaa6 is any amino acid
XXXXXX

SEQ ID NO: 8 - DNA, 21 bp, Artificial Sequence
  5-therminal thiol modified GAPDH siRNA purchased from Genepharm Shanghai
guaugacaacagccucaagtt

SEQ ID NO: 9 - DNA, 21 bp, Artificial Sequence
  5-terminal thiol modified GAPDH siRNA purchased from Genepharm Shanghai
cuugaggcuguugucauactt